(19) United States
(12) Patent Application Publication
(10) Pub. No.: US 2017/0015642 A1
(43) Pub. Date: Jan. 19, 2017

(54) CONVERSION OF FRUCTOSE-CONTAINING FEEDSTOCKS TO HMF-CONTAINING PRODUCT

(71) Applicant: RENNOVIA, INC., Santa Clara, CA (US)

(72) Inventors: Valery Sokolovskii, Santa Clara, CA (US); Eric L. Dias, Belmont, CA (US); Hong X. Jiang, Palo Alto, CA (US); James M. Longmire, San Jose, CA (US); Vincent J. Murphy, San Jose, CA (US); Christopher Paul Dunckley, San Jose, CA (US); Gary M. Diamond, Menlo Park, CA (US); Thomas R. Boussie, Menlo Park, CA (US); James A.W. Shoemaker, Gilroy, CA (US); Liza Lopez Soto, Fremont, CA (US)

(73) Assignee: RENNOVIA, INC., Santa Clara, CA (US)

(21) Appl. No.: 15/132,178

(22) Filed: Apr. 18, 2016

Related U.S. Application Data
(63) Continuation-in-part of application No. 14/606,789, filed on Jan. 27, 2015.
(60) Provisional application No. 61/932,185, filed on Jan. 27, 2014.

(51) Int. Cl. C07D 307/46; B01D 61/14; C07D 307/42

(52) U.S. Cl. CPC C07D 307/46; C07D 307/42; B01D 61/145

(57) ABSTRACT

The present invention relates generally to processes for converting fructose-containing feedstocks to a product comprising 5-(hydroxymethyl)furfural (HMF) and water in the presence of water, solvent and an acid catalyst. In some embodiments, the conversion of fructose to HMF is controlled at a partial conversion endpoint characterized by a yield of HMF from fructose that does not exceed about 80 mol %. In these and other embodiments, the processes provide separation techniques for separating and recovering the product, unconverted fructose, solvent and acid catalyst to enable the effective recovery and reutilization of reaction components.

### CROSS REFERENCE TO RELATED APPLICATIONS

This application is a continuation-in-part of U.S. application Ser. No. 14/606,789, filed on Jan. 27, 2015, which claims benefit of U.S. provisional application Ser. No. 61/932,185, filed Jan. 27, 2014, the entire disclosure of which is incorporated herein by reference.

### FIELD OF THE INVENTION

The present invention relates generally to processes for converting fructose-containing feedstocks, for example, high fructose corn syrup-containing feedstocks, to a product comprising 5-(hydroxymethyl)furfural (HMF) and water. In one aspect of the invention, the process comprises the step of converting a fructose-containing feedstock to HMF in a reaction zone in the presence of water, solvent and acid catalyst to attain a relatively low specified yield of HMF at a partial conversion endpoint and thereafter the conversion of fructose to HMF is quenched at the partial conversion endpoint. Typically, the sum of unconverted fructose, HMF yield, and the yield of intermediates is at least 90 mol % at the partial conversion endpoint. In another aspect of the invention, the process comprises partially converting the feedstock in a reaction zone in the presence of water, solvent and an acid catalyst, removing from the reaction zone the combination resulting from the partial conversion, separating unconverted fructose from the reaction combination removed from the reaction zone, and separating solvent separately from the separation of the unconverted fructose, the separations being conducted to enable the subsequent recovery of product comprising HMF and water. The post reaction zone separations also enable the effective recovery and reutilization of unconverted fructose and solvent. In another aspect of the invention, selective membrane separation techniques are employed for the separation and recovery of unconverted fructose and intermediates from the desired product.

### BACKGROUND OF THE INVENTION

HMF has been recognized as a chemical with potentially significant industrial and commercial applications because of its high degree of functionality and its ability to act as a precursor to various industrially useful chemicals. See Werpy, T; Petersen, G. (Eds.), “Top Value Added Chemicals from Biomass, Vol. 1: Results of Screening for Potential Candidates from Sugars and Synthesis Gas,” U.S. Dept. of Energy, Office of Scientific Information: Oak Ridge, Tenn. DOE/GO-102004-1992 (2004). For example, its functionality affords use in the production of solvents, surfactants, pharmaceuticals and plant protecting agents, and furan derivatives thereof which are useful as monomers for the preparation of non-petroleum derived polymers.

HMF is primarily produced by dehydrating a carbohydrate feedstock, particularly monosaccharides such as glucose and fructose. Complications commonly arise during the reaction as a result of the production of unwanted acid by-products, particularly levulinic and formic acid, and especially the polymerization of reaction components which forms humins (a mixture of colored, soluble and insoluble oligomers and polymers), all of which reduce the overall process yield and complicate the recovery of HMF, making large scale production of HMF economically unattractive. These complications are exacerbated by the desire to maximize conversion of feedstock to HMF in the reaction zone.

Fructose is the preferred hexose to produce HMF because it has been demonstrated to be more amenable to dehydration reactions than other hexoses including glucose. High fructose corn syrup (HFCS) is a high volume, commercially available product from which HMF and other furans could be produced in large quantities. Currently, as much as 18 billion pounds/yr of high fructose corn syrup are produced. Szmant et al, J. Chem. Tech. Biotechnology, Vol. 31, PP 135-45 (1981) disclosed the use of high fructose corn syrup as a feedstock for the production of HMF.

A variety of homogeneous catalysts have been employed to promote the dehydration of fructose to HMF. Inexpensive strong inorganic acids have been used: see, for example, U.S. Pat. No. 7,572,925. Organic acids have also been disclosed, including relatively strong organic acids such as p-toluene sulfonic acid and weaker organic acids such as oxalic acid and levulinic acid: See, for example, U.S. Pat. No. 4,740,605, which discloses oxalic acid. All patents and other publications cited in this application are incorporated herein by reference.

Similarly, a variety of heterogeneous catalysts have been reported as useful for the dehydration of carbohydrate to HMF. See, for example de Vries, Chem. Rev. 2013, pp 1499-1597. Dumesic, ACS Catal 2012, 2, pp 1865-1876; and Sandborn, U.S. Pat. No. 8,058,458. Fleche, in U.S. Pat. No. 4,339,387, disclosed the use of solid acid resin catalysts where the resin may be a strong or weak cationic exchanger, with the functionalization preferably being in the H⁺ form (including, for example, resins under the trademark Amberlite C200 from Rohm & Haas Corporation and Lewatit SPC 108 from Bayer AG). Sanborn, in AU 2011205116, disclosed that metals such as Zn, Al, Cr, Ti, Th, Zr and V are useful as catalysts. And Binder, in US 2010/0004437 A1, disclosed the use of a halide salt.

In addition to the use of catalysts in the dehydration of carbohydrates to HMF, there has been much focus on solvents and solvent systems that reportedly are beneficial in the process. See for example, de Vries Chem. Rev 2013, 113, 1499-1597.

A multitude of processes have been disclosed for the production of HMF from fructose. However, the known prior processes have not recognized any benefit associated with low conversion in the reaction zone. Typically, research has focused on attaining the highest possible conversion of fructose to HMF in the reaction zone, which inevitably has resulted in increased off-path products, including humins, and/or process complexity and expense. In the quest to attain high conversion of fructose to HMF in the reaction zone, prior processes have focused on improving catalyst performance, reactor solvent systems and reactant mixing techniques, using solvent modifiers to improve phase separations in the reactor, using foam and/or oxidation suppressants, reducing carbohydrate concentration in the reactor, using very high temperatures and/or pressures, and performing multiple steps in the reactor (e.g., steam injection or controlled vaporization to simultaneously remove certain constituents), among other techniques. Nevertheless, none of the processes disclosed to date appears to have overcome the low overall process productivity in a commercially economically viable manner.

In order to overcome the shortcomings of the prior processes, applicants have discovered processes based upon intentionally limiting the conversion of fructose to HMF in the reaction zone. In these processes, HMF, unconverted fructose, solvent and, when applicable, catalyst are removed from the reaction zone and ultimately separated from one another, enabling the efficient recycling of these separated constituents and, ultimately, the cost effective production and recovery of large quantities of HMF.

### SUMMARY OF THE INVENTION

Briefly, therefore, the present invention is directed to improved processes for converting fructose-containing feedstocks to a product comprising HMF and water.

In one embodiment, the process comprises combining fructose, water, an acid catalyst and a first solvent in a reaction zone and converting in the reaction zone fructose to HMF and water and to intermediates to HMF to a partial conversion endpoint. The yield of HMF from fructose at the partial conversion endpoint does not exceed about 80 mol %. At least a portion of the product, unconverted fructose and the first solvent are removed from the reaction zone, as a combination, wherein the conversion of fructose to HMF in the combination removed from the reaction zone is quenched at the partial conversion endpoint. At least a portion of each of the first solvent, the product and unconverted fructose in the combination removed from the reaction zone are separated from one another. At least a portion of the separated unconverted fructose and at least a portion of the separated first solvent are subsequently recycled to the reaction zone and the product comprising HMF and water is recovered.

In accordance with another embodiment, the process comprises combining fructose, water, an acid catalyst and at least a first solvent in a reaction zone and converting in the reaction zone a portion of the fructose to HMF and water. At least a portion of the product, unconverted fructose and the first solvent are removed from the reaction zone as a combination and at least a portion of the combination is contacted with a second solvent in a fructose separator to separate at least a portion of unconverted fructose from the combination and produce an intermediate composition having a reduced fructose concentration and comprising the product and at least a portion of each of the first solvent and second solvent. At least a portion of the separated, unconverted fructose is recovered and at least a portion of the first solvent, the second solvent and the product in the intermediate composition are separated from one another.

In accordance with a further embodiment, the process comprises combining fructose, water, an acid catalyst and at least a first solvent in a reaction zone and converting in the reaction zone a portion of the fructose to HMF and water and to intermediates to HMF. At least a portion of the product, unconverted fructose, intermediates and first solvent are removed from the reaction zone as a combination and one or more constituents of the combination withdrawn from reaction zone are separated by selective membrane separation.

Other objects and features will be in part apparent and in part pointed out hereinafter.

### BRIEF DESCRIPTION OF THE DRAWINGS

FIG. 1 graphically illustrates a typical conversion of fructose to HMF in a reaction zone as a function of time, highlighting changes in fructose, HMF and intermediate concentrations as well as changes in reaction mass balance, the latter of which is reflective of an increased concentration of off-path reaction products (including humins) at higher fructose conversions.

FIG. 2 depicts an example of a process flow diagram illustrating certain aspects of the present invention associated with the partial conversion of the fructose-containing feedstock to HMF, including separate solvent and unconverted fructose separation steps, recovery of catalyst (when applicable) and recycling of some or all of these constituents to the reaction zone or elsewhere.

FIG. 3 depicts an example of a process flow diagram of a process employing chromatographic separations technology (e.g., simulated moving bed technology) to effect separation of unconverted fructose and intermediates from the product comprised of HMF and water.

FIG. 4 depicts an example of a process flow diagram of a process wherein a liquid-liquid extraction step is employed to separate initially, and downstream of the reaction zone, at least a portion of the unconverted fructose and intermediates from the combination withdrawn from the reaction zone.

FIG. 5 depicts an example of a process flow diagram of a process wherein a liquid-liquid extraction step is employed to separate initially, and downstream of the reaction zone, at least a portion of the unconverted fructose and intermediates and wherein a second solvent is added downstream of the reaction zone to effect improved partitioning of HMF from unconverted fructose.

FIG. 6 depicts an example of a process flow diagram of an alternative process configuration employing a liquid-liquid extraction step wherein a polar solvent and non-polar solvent are added to the reaction zone and the polar solvent is removed prior to a liquid-liquid extraction step to enable partitioning of HMF from unconverted fructose.

FIG. 7 depicts an example of a process flow diagram of a further alternative process configuration employing two solvents, one of which is employed to provide enhanced partitioning in liquid-liquid extraction to enable portioning of HMF from unconverted fructose.

FIG. 8 depicts an example of a process flow diagram of a process configuration employing the use of ultra-filtration and nano-filtration to enable the separation of HMF from unconverted fructose and intermediates.

FIG. 9 graphically illustrates the conversion of fructose to HMF in a continuous flow reaction zone as a function of HCl concentration at a fixed residence time, highlighting changes in fructose, HMF and intermediates concentrations.

FIG. 10 shows the results of the experiment presented in Example 9, in which five different alumina samples were tested for their capacity to remove humins from the product effluent (organic solvent/water mixture solution) resulting from conversion of fructose to HMF.

FIG. 11 shows an HLPC trace illustrating alumina sasol 1.8/210 removal of HAF contaminants from the product effluent (organic solvent/water mixture solution) resulting from conversion of fructose to HMF.

FIG. 12 graphically illustrates the removal of reaction by-products from an product effluent (organic solvent/water mixture solution) resulting from conversion of fructose to HMF, as a function of alumina surface area. The changes in fructose, HMF and intermediate concentrations are highlighted.

FIG. 13 shows the results of a desorption experiment in which humins were desorbed with an aqueous solution of 0.1N NaOH.

Corresponding reference characters indicate corresponding parts throughout the drawings.

### DESCRIPTION OF THE PREFERRED EMBODIMENTS

In accordance with the present invention, any of a variety of fructose-containing feedstocks can be employed including, without limitation, essentially pure fructose, sucrose, mixtures of glucose and fructose, and combinations thereof. Moreover, the present invention contemplates the use of starches, cellulosics and other forms of carbohydrates which, for example, are subjected to processing conditions that isomerize glucose produced from the starches or cellulosics to form fructose-containing feedstocks.

An aspect of the present invention is the partial conversion of a fructose-containing feedstock to HMF. The conversion is carried out in a reaction zone that contains at least fructose-containing feedstock, water, acid catalyst and solvent.

Water can be present in a reaction zone either as a separately added constituent or as a component of, for example, a solution of fructose-containing feedstock. Conjunctively or alternatively, and without limiting the scope of the invention, water may be present in a reaction zone as a solution comprised of a reaction modifier, such as an aqueous salt solution, as more fully described hereinafter.

Typically, an aqueous solution of fructose is used as the feedstock to the reaction zone. In various preferred embodiments, commercially available high fructose corn syrup (HFCS) is dissolved in water to form the solution. For example, HFCS-97 or HFCS-90 may be used.

The concentration of fructose in a reaction zone is generally in the range of from about 5 wt % to about 80 wt % dissolved solids. In various embodiments, the concentration of dissolved solids is in the range of about 20 wt % to about 80 wt %. In various embodiments, the concentration of dissolved solids is at least about 40 wt %. In some embodiments, it may be desirable to lower the concentration of fructose in the solutions to 20 wt % or less.

In accordance with the present invention the reaction takes place in a reaction zone in the presence of an acid catalyst. The catalyst may be a homogeneous or heterogeneous catalyst. Homogeneous catalysts include Brønsted or Lewis acids. Examples of such acids include organic and inorganic acids. Inorganic acids include mineral acids and other strong acids. Brønsted acids include HCl, HI, H₂SO₄, HNO₃, H₃PO₄, oxalic acid CF₃SO₃H and CH₃SO₃H. Lewis acids can include for example, borontrihalides, organoboranes, aluminum trihalides, phosphorus and antimony pentafluorides, rare earth metal triflates, and metal cation ether complexes. Preferred acids are Brønsted acids selected from the group of HCl, HBr, H₂SO₄ and H₃PO₄. Quantities of catalyst when homogeneous are typically in the range of from about 0.1 to about 25 mol. % vs. hexose, more typically from about 0.5 to about 10 mol. % or from about 0.5 to about 5 mol. %. Suitable heterogeneous catalysts include acid-functionalized resins, acidified carbons, zeolites, micro- and meso-porous metal oxides, sulfonated and phosphonated metal oxides, clays, polyoxometallates and combinations thereof. Preferred heterogeneous catalysts include acid functionalized resins. When a heterogeneous catalyst is employed, the catalyst loading in the reaction mixture will depend upon the type of reactor utilized. For example, in a slurry reactor, the catalyst loading may range from about 1 g/L to about 20 g/L; in a fixed bed reactor the catalyst loading may range from about 200 g/L to about 1500 g/L.

Also present in the reaction zone is a solvent. Solvents are typically organic solvents and can either be polar or non-polar solvents. Generally, useful solvents can be selected from among ethers, alcohols, ketones and hydrocarbons. Examples of useful solvents include ethers such diethyl ether, methyl tert-butyl ether, dimethoxyethane (DME or glyme), bis(2-methoxyethyl) ether (diglyme), tetrahydrofuran (THF), dioxane, and 2-methyltetrahydrofuran (MeTHF), ketones such as acetone, methyl ethyl ketone and methyl isobutyl ketone (MIBK), alcohols such as isopropanol, 2-butanol, and tert-butanol, and hydrocarbons such as pentane, hexane, cyclohexane and toluene. In various embodiments, solvents include DME, dioxane, THF, MeTHF, 2-butanol, and MIBK.

The fructose-containing feedstock, water, catalyst and solvent can exist in the reaction zone as a mono- or multi-phasic system. The amount of solvent in the system relative to water typically ranges from 10:1 to 1:1 on a mass basis. In various embodiments it can range from 5:1 to 2:1. The presence of organic solvent in the reaction zone promotes both faster reaction rates and higher yields of HMF. Solvent-water combinations that form either mono- or multi-phasic compositions in the reaction zone can be employed. Preferred solvents for the reaction zone are unreactive under the conditions of fructose dehydration, and have boiling points lower than water.

An important aspect of the invention is the partial conversion of the fructose in the reaction zone. That is, the dehydration reaction is allowed to proceed until a partial conversion endpoint is attained and then the reaction is at least partially quenched (i.e., the conversion of fructose is reduced). In accordance with the present invention, the conversion of fructose in the reaction zone is controlled such that at the partial conversion endpoint, the yield of HMF from fructose provided to the reaction zone is maintained at a relatively low specified yield. As discussed in greater detail below, applicants have discovered that controlling the conversion of fructose to HMF at a specified yield reduces conversion of HMF and/or fructose to off-path products such as oligomers and polymers produced from the reaction components and referred to herein as humins, especially those which are soluble in water or the solvent supplied to the reaction zone.

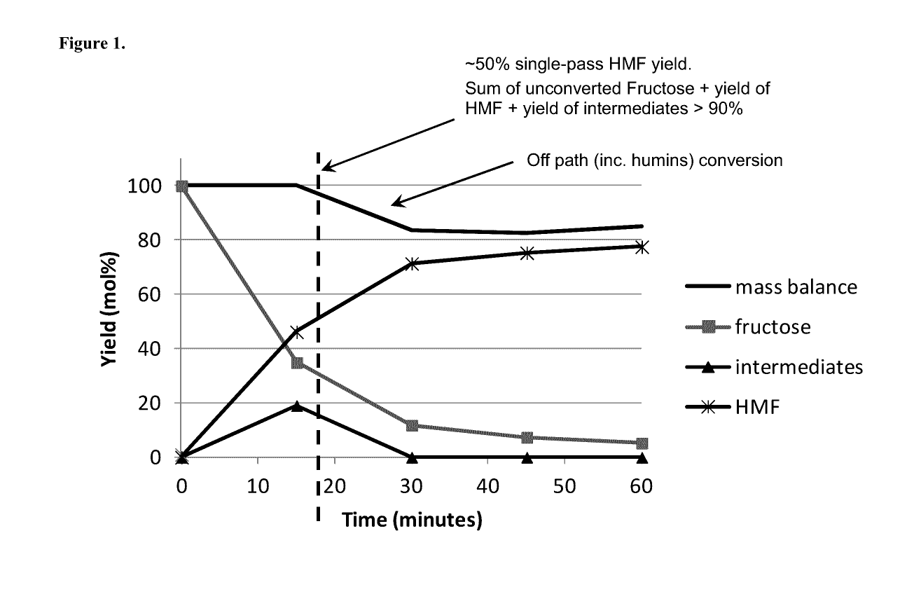

FIG. 1 graphically illustrates a typical conversion of fructose to HMF in a reaction zone as a function of time, highlighting changes in fructose, HMF and intermediate concentrations as well as changes in reaction mass balance, the latter of which is reflective of an increased concentration of off-path reaction products (e.g., levulinic acid, formic acid, and soluble and insoluble humins) at higher fructose conversions. Mass balance in this instance is defined as the sum of unconverted fructose plus the mol % yield of HMF plus the mol % yield of reaction intermediates. As discussed by István T Horvath et al. (Molecular Mapping of the Acid-Catlaysed Dehydration of Fructose, Chem. Commun., 2012, 48, 5850-5852), several different reaction pathways exist for the conversion of fructose to HMF as well as the generation of various off-path products that are believed to lead to the formation of humins. On-path intermediates to HMF are reported to include isomers of fructose such as α-D-fructofuranose and β-D-fructofuranose, 2,6-anhydro-β-D-fructofuranose, fructofuranosyl oxocarbenium ions, (2R,3S,4S)-2-(hydroxymethyl)-5-(hydroxyl-methylene)-tetrahydrofuran-3,4-diol, (4S,5R)-4-hydroxy-5-hydroxymethyl-4,5-dihydrofuran-2-carbaldehyde and difructose dianhydrides (DFAs). Off-path intermediates are reported to include (3S,4R,5R)-2-(hydroxymethylene)-tetrahydro-2H-pyran-3,4,5-triol and (3R,4S)-3,4-dihydroxy-3,4-dihydro-2H-pyran-6-carbaldehyde, which can be converted to humins.

FIG. 1 also graphically depicts a typical conversion of fructose-containing feedstock to HMF in accordance with the present invention, highlighting certain of the benefits attributable to partial conversion to HMF. More specifically, at time zero, no conversion occurs. At time “t” (represented by the dashed line extending parallel to the yield axis) a 50% molar yield of HMF is produced through conversion of fructose in the feedstock (as indicated by the intersection of the dashed line with the HMF yield line). Also, at time “t”, the concentration of fructose is significantly reduced (to about 30 to about 35% of the starting concentration). Further, at time “t” in this example, intermediates formation has effectively peaked. As to the formation of off-path product, including humins, applicants have discovered that at a partial conversion of fructose to HMF characterized by a relatively low specified yield of HMF (for example, as shown in FIG. 1 where the yield of HMF is about 50% or less at time “t”), the reaction to these undesired products is significantly reduced, as illustrated by the mass balance being >90%. Generally, off-path product at the partial conversion endpoint is maintained at not more than about 10%, more typically not more than about 8%, in various embodiments does not exceed about 5% (as illustrated in FIG. 1), and in various preferred embodiments can be controlled so as not to exceed about 3%. Thus, in one aspect of the invention the sum of unconverted fructose, the yield of HMF from fructose and the yield of intermediates at the partial conversion endpoint should be at least about 90%, in various embodiments at least about 92%, more typically at least about 95% and in various preferred embodiments at least about 97%.

As demonstrated in Example 7, the specified yield of HMF at the partial conversion endpoint can be suitably increased above 50% and still attain the desired benefits of reduced production of off-path intermediates and improved overall process yield of HMF. More particularly, in accordance with the present invention, the conversion of fructose in the reaction zone is controlled such that at the partial conversion endpoint, the yield of HMF from fructose provided to the reaction zone is not more than about 80%, not more than about 75%, not more than about 70%, not more than about 65%, not more than about 60%, not more than about 55% or not more than about 50%. For economic reasons, the yield of HMF in the reaction zone at the partial conversion endpoint is generally not less than about 30% and typically not less than about 40%. Thus, the yield of HMF from fructose provided to the reaction zone at the partial conversion endpoint is generally controlled at from about 30 to about 80%, from about 30 to about 75%, from about 30 to about 70%, from about 30 to about 65%, from about 30 to about 60%, from about 30 to about 55%, from about 30 to about 50%, from about 40 to about 80%, from about 40 to about 75%, from about 40 to about 70%, from about 40 to about 65%, from about 40 to about 60%, from about 40 to about 55%, from about 40 to about 50% or from about 40 to about 45%. On the other hand, the upper end of the HMF yield at the partial conversion endpoint will depend on various factors, including the nature and concentration of the catalyst, water concentration, solvent selection and other factors that can influence the generation of off-path products. Generally, operation within the ranges for HMF yield at the partial conversion endpoint as disclosed herein are consistent with the adequate control of the production of off-path intermediates while maintaining desired overall process yield of HMF.

In accordance with various embodiments of the invention, to effect partial conversion, the reaction zone is generally maintained at a temperature in the range of from about 50° C. to about 250° C., more typically in the range of from about 80° C. to about 180° C. In some embodiments, the reaction zone is maintained at a temperature in the range of from about 100° C. to about 160° C., or in the range of from about 100° C. to about 150° C., or in the range of about 100° C. to about 140° C., or in the range of from about 110° C. to about 130° C. Generally, higher temperatures increase the reaction rate and shorten the residence time necessary to reach the partial conversion endpoint. The reaction constituents within the reaction zone are typically well-mixed to enhance the conversion rate and the zone is typically maintained at a pressure in the range of from about 1 atm to about 15 atm or from about 2 atm to about 10 atm. In various embodiments, the temperature and pressure within the reaction zone are maintained such that the constituents in the reaction zone are largely maintained in the liquid phase. The pressure in the reaction zone can be maintained by supplying an inert gas such as nitrogen.

The time during which the reaction is carried out in the reaction zone prior to the partial conversion endpoint and before quenching the conversion of fructose and removal of materials from the zone is variable depending upon the specific reaction conditions employed (e.g., reaction temperature, the nature and quantity of the catalyst, solvent selection, water concentration in the reaction zone, etc.) and generally can range from about 1 to about 60 minutes. The composition of the reaction mixture with respect to HMF yield from fructose and the concentration of intermediates to HMF from fructose and of unconverted fructose can be monitored using various means known to those skilled in the art to determine and establish the desired partial conversion endpoint in accordance with the present invention. For example, periodic sampling and analysis (e.g., by HPLC) of the reaction zone materials is but one of several ways to determine and establish the partial conversion endpoint. Additionally or alternatively, the composition of the reaction mixture may be monitored using the dehydration reaction mass balance, wherein a decrease in the mass balance is reflective of an increased concentration of off-path reaction products (including humins) and thus a commensurate decrease in the sum of unconverted fructose, the yield of HMF from fructose and the yield of intermediates. The partial endpoint control method can be integrated into a programmed process control scheme based on an algorithm generated using historical analytical data, and can be updated by on-line or off-line analytical data.

Once the desired partial conversion endpoint is attained, the dehydration reaction and conversion of fructose is typically at least partially quenched to avoid significant additional production of any off-path products (e.g., levulinic acid, formic acid, and soluble and insoluble humins). Typically, at least a portion of the combination produced in the reaction zone is withdrawn for subsequent processing and product recovery as described in detail below. In these and other embodiments, the conversion of fructose can be suitably quenched after the partial conversion endpoint is attained by reducing the temperature of the reaction constituents either within the reaction zone or after being withdrawn from the zone using various industrial means known to those skilled in the art. For example, and without limitation, the reaction constituents may be cooled by flash evaporation, contact with a cooling inert gas, mixing with a liquid diluent, passage through an indirect heat exchanger or a combination of these and other techniques. Typically, in such embodiments, the reaction constituents are cooled to a temperature below about 100° C., more typically, below about 60 or 50° C. It should be understood that other means for quenching the conversion of fructose may be employed without departing from the present invention. For example, in embodiments where a heterogeneous catalyst that is retained in the reaction zone (e.g., a fixed bed catalyst) is employed, the conversion of fructose at the partial conversion endpoint can be quenched by withdrawing some or all of the combination produced from the reaction zone.

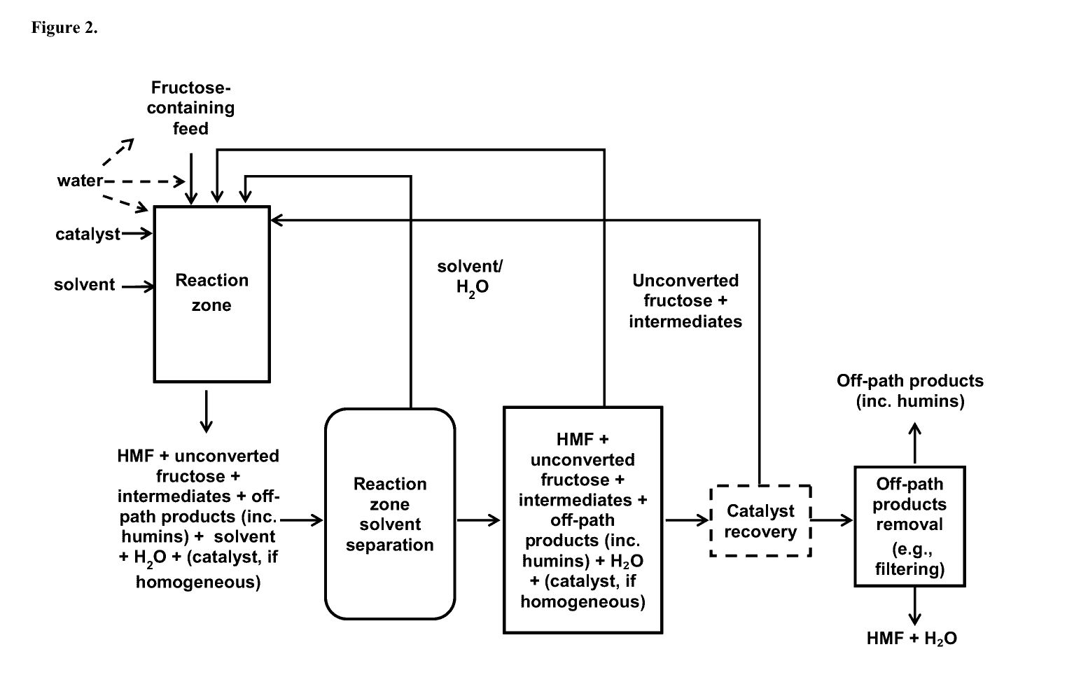

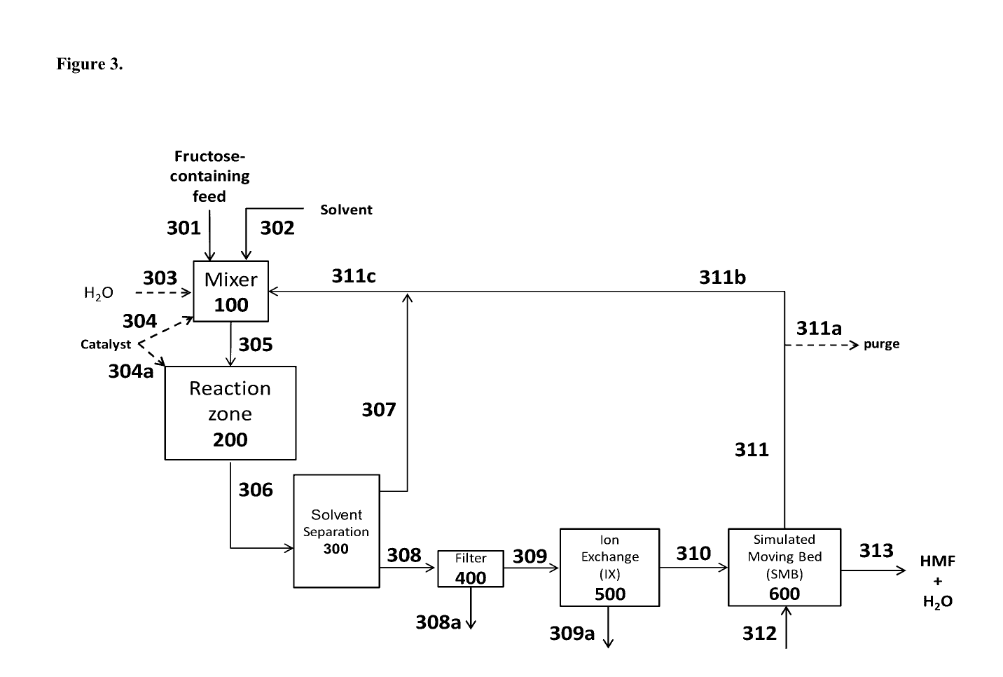

FIG. 2 illustrates basic process steps employed for the partial conversion of fructose-containing feedstocks to HMF in accordance with the present invention. As illustrated in FIG. 2, feedstock is added as an aqueous solution to the reaction zone, or feedstock and water may be added separately. Additionally, catalyst (heterogeneous or homogeneous) is added to the reaction zone. In the case of a heterogeneous catalyst, the catalyst is typically added to the reaction zone prior to the addition of the feedstock, water and solvent. In the case of a homogeneous catalyst, the catalyst may be pre-mixed with the feedstock and/or solvent before being supplied to the reaction zone (see FIG. 3 et seq.) or may be added before, simultaneously with or after the feedstock, water and/or solvent is added to the reaction zone. Further, solvent may be added to the reaction zone before, simultaneously with or after addition to the reaction zone of one or more of the other reaction zone constituents. Again, in various embodiments of the present invention, regardless of the order in which the constituents are provided to the reaction zone, some or all of the reaction constituents may be mixed prior to addition to the reaction zone or mixed in the reaction zone, all so as to enhance the conversion rate in the reaction zone. Mixing can be undertaken by any of a variety of means well known in the art.

In accordance with the present invention, the conversion step can be carried out in one or more reaction zones. For illustrative purposes, the figures depict only one reaction zone. The process may be carried out in batch, semi-continuously or substantially continuous manner. Any of a variety of well known reactor designs defining at least one reaction zone is suitable for carrying out the process of the present invention. For example, and without limitation, useful reactors include tank reactors, continuously stirred tank reactors (CSTRs), flow through continuous reactors, fixed bed continuous reactors, slurry type reactors and loop reactors, among others. Single reactors may be employed or combinations of several reactors. Again, reactors may comprise one or more reaction zones. Multiple reaction zones in series may be employed using, for example, cascading tank reactors or continuous reactors, or one continuous reactor provided with multiple, separated reaction zones. Those of ordinary skill in the art will appreciate the multitude of reactor configurations which may be employed to achieve the objectives of the present invention.

The output from the reaction zone is a combination comprising HMF, unconverted fructose, intermediates produced during the conversion step, solvent, water and off-path products which may result from the conversion step. Additionally, when homogeneous catalyst is employed, the output from the reactor will include catalyst. Output from the reactor (i.e., the combination removed from the reaction zone at the partial conversion endpoint) includes, quantitatively, at least some amount of each constituent provided to the reaction zone (excluding catalyst, other than impurity amounts, in embodiments in which fixed bed heterogeneous catalysts are employed). For example, in an embodiment employing a tank reactor, the entire contents of the reactor (again, the combination) may be removed after the partial conversion endpoint is attained. Alternatively, for example, in embodiments employing continuous flow reactors, only a portion of the contents in the reaction zone (again, the combination) may be removed in a given period of time to establish a minimum reactor residence time necessary to attain a target partial conversion endpoint.

FIG. 3 illustrates an embodiment of the partial conversion process of the present invention using a homogeneous catalyst and employing a combination of a solvent separator 300, a catalyst recovery unit 500, and a product recovery unit 600 to separate and remove unconverted fructose and intermediates from the desired product, HMF in water, and enable recycling of certain reaction constituents. In this embodiment, an aqueous stream of fructose-containing feedstock is supplied via 301 to mixer 100 for mixing reaction constituents (e.g., a stirred tank). Also provided to mixer 100 via 302 is fresh and make up solvent, water via 303, and catalyst via 304. In this embodiment, catalyst may also be provided to a reaction zone 200 via 304a. As contemplated in FIG. 3, supply of catalyst to mixer 100 and reaction zone 200 need not be exclusive to either; instead, it may be supplied to both. The mixed reaction constituents are supplied to the reaction zone via 305. In the reaction zone 200, fructose is converted to HMF until the partial conversion endpoint is attained and then the conversion reaction is suitably quenched as described above. At least a portion of the reaction constituents, product (HMF and water), intermediates to HMF, solvent (in this embodiment the solvent is preferably polar) and off-path products (such as levulinic acid, formic acid, and soluble and insoluble humins, among others) are removed from the reaction zone as a combination and supplied via 306 to solvent separator 300 for separating at least a portion of solvent from the combination. In embodiments where the boiling point of the solvent is significantly lower than the other components of the combination, a simple evaporative separation may be carried out and the heat of vaporization may optionally be used to cool the reaction components in quenching the conversion of fructose. However, in embodiments where, for example, the boiling point of the solvent is relatively close to (whether above or below) that of other components of the combination, a distillation unit may be utilized wherein a fraction composed substantially of solvent and some water, preferably essentially only solvent, can be withdrawn at an appropriate location along the length of the column. Separated solvent is typically condensed to a liquid phase and preferably, as illustrated for example in FIG. 3, supplied via 307 as a component of the recycled mixture provided to the mixer 100 via 311c. In various embodiments, partial solvent separation is preferred as it may be advantageous in assisting the separation of fructose from the product.

The remaining constituents from the combination withdrawn from reaction zone 200 are delivered via 308 to a filtration unit 400. In filtration unit 400 insoluble, typically solid, humins are removed from the stream 308 and disposed of via 308a. The remaining liquid from filtration unit 400 is delivered via 309 to catalyst recovery unit 500 (e.g., an ion exchange unit) designed, for example when HCl or H₂SO₄ is the catalyst, to capture the chloride or sulfate ions on the exchange resin prior to the separation of the unconverted fructose from the product. The “catalyst free” eluent from the catalyst recovery unit 500 is supplied via 310 to product recovery unit 600, which in the illustrated embodiment is a continuous chromatographic separation (e.g., simulated moving bed, liquid chromatography or, for short, SMB) unit in which the typically more difficult separation of the unconverted fructose from the product is carried out. SMB units are well known to those of ordinary skill in the art of separations; for example, SMB units are industrially employed in the separation of similar products such as, for example, glucose from fructose. In operation, water is added to the bed via 312 and the mixture of HMF, unconverted fructose and water flows through the multiple columns of the SMB unit to separate HMF from fructose. Ultimately, not more than about 10%, typically not more than about 5%, or not more than about 2% of the unconverted fructose is unseparated from the HMF. The product is removed via 313 and the unconverted fructose is removed via 311. Optionally, a purge stream 311a is provided to remove some of the collected, unconverted fructose and water for any of a variety of purposes including, for example, testing, use in another reaction train, to maintain process water balance or for other purposes. The remainder, stream 311b, can be combined with recovered solvent from stream 307 and resupplied to mixer 100 ultimately as a constituent of recycle stream 311c.

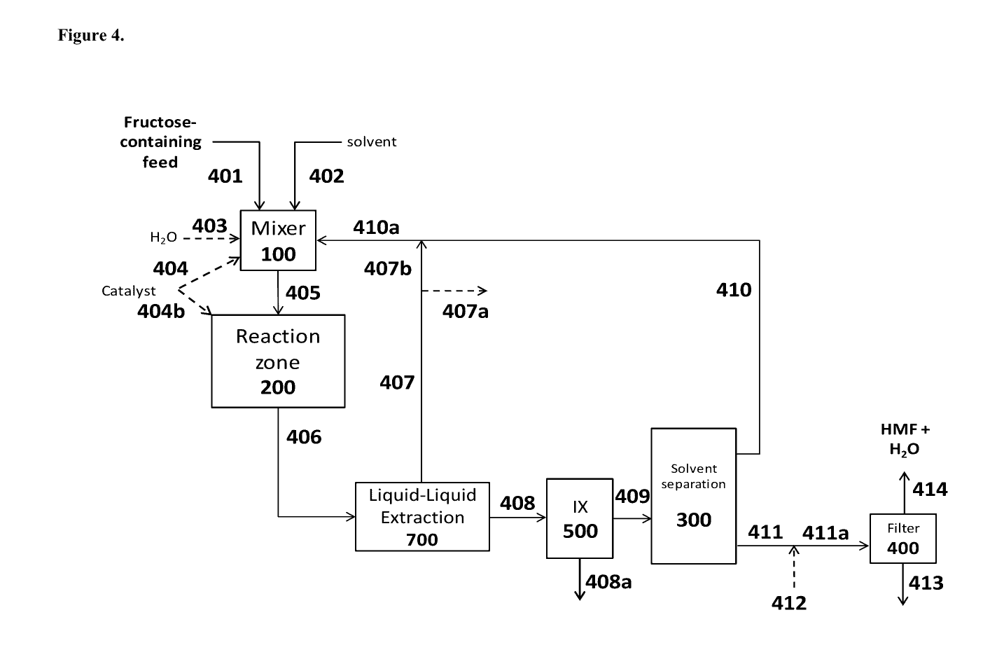

FIG. 4 illustrates an embodiment of the partial conversion process of the present invention using a homogeneous catalyst and employing a combination of a fructose separator 700 for separating unconverted fructose from the combination removed from the reaction zone, for example, by employing liquid-liquid extraction technology, a catalyst recovery unit 500, a solvent separator 300, and a filter 400 for removing off-path products such as insoluble humins from product. In this embodiment, an aqueous stream of fructose-containing feedstock is supplied via 401 to mixer 100 for mixing reaction constituents (e.g., a stirred tank). Also provided to mixer 100 via 402 is fresh and make up solvent, water provided via 403, and catalyst via 404. In this embodiment, catalyst may also be provided to a reaction zone 200 via 404b. As contemplated in FIG. 4, supply of catalyst to mixer 100 and reaction zone 200 need not be exclusive to either; instead, it may be supplied to both. The mixed reaction constituents are supplied to the reaction zone via 405. In the reaction zone 200, fructose is converted to HMF until the partial conversion endpoint is attained and then the conversion reaction is suitably quenched as described above. At least a portion of the reaction constituents, product (HMF and water), intermediates to HMF, solvent (in this embodiment the solvent may be polar or non-polar, preferably polar) and off-path products (such as levulinic acid, formic acid, and soluble and insoluble humins, among others) are removed from the reaction zone in combination and supplied via 406 to fructose separator 700 for separating unconverted fructose from the combination removed from the reaction zone.

In one embodiment, fructose separator 700 is a liquid-liquid extraction apparatus. This separation method is well known and encompasses establishing conditions that enable partitioning of one or more constituents into one layer (phase) preferentially as compared to another layer (phase) that forms in the vessel as a result of conditions established therein. Partitioning can be achieved by, for example, choosing an appropriate solvent or by adding to fructose separator 700 a composition of matter that promotes the partitioning. It has been proposed in US 2010/0004437 A1 that unconverted fructose can be extracted from a reaction product comprised of HMF, solvent and water by adding salts such as for example NaCl or MgCl₂. In some embodiments, the solvent used to extract unconverted fructose can be used as a cooling medium to quench the conversion of fructose.

An unexpected advantage of embodiments of the present invention in which liquid-liquid separation is employed is that the homogeneous acid catalyst is readily recovered and easily resupplied to the reaction zone with, for example, the unconverted fructose. The partitioned unconverted fructose and at least a portion of the acid catalyst are removed via 407. A part of the partitioned unconverted fructose may optionally be purged via 407a for any of a variety of reasons. For example, a portion of the water that may have been partitioned with the unconverted fructose may be separated, for example, by using an evaporator and the unconverted fructose with reduced water content returned to the reaction zone to maintain water balance. Ultimately, not more than about 10%, typically not more than about 5%, or not more than about 2% of the unconverted fructose remains in the liquid fed via 408 to catalyst recovery unit 500.

The remaining constituents partitioned in the other layer (in this embodiment comprising product, catalyst, any partitioning additive and solvent are delivered via 408 to catalyst recovery unit 500 (e.g., an ion exchange unit) designed, for example when HCl or H₂SO₄ is the catalyst, the capture the residual chloride or sulfate ions on the exchange resin prior to isolation of the product. In this embodiment it is anticipated that at least a portion, more preferably essentially all, of the homogeneous catalyst is separated during the liquid-liquid extraction process. The catalyst is separated into the phase containing the unconverted fructose and consequently may be recovered and recycled to the reaction zone. The “catalyst free” eluent from the ion exchange unit 500 is supplied via 409 to the solvent separator 300 for separating solvent(s) from the remaining constituents of the combination. In embodiments where the boiling point of the solvent is significantly lower than the other components of the combination, a simple evaporative separation may be carried out; however, in embodiments where, for example, the boiling point of the solvent is relatively close to (whether above or below) that of other components of the combination, a distillation unit may be utilized wherein a fraction composed substantially of solvent and some water, preferably essentially only solvent, can be withdrawn at an appropriate location along the length of the column. Separated solvent is preferably, as illustrated in FIG. 4, supplied via 410 as a component of the recycled mixture provided to the mixer 100 via 410a. The remaining constituents from the combination withdrawn from the solvent separator 300 via means 411 are delivered via 411a, optionally with additional water supplied via 412, to filter 400. In filter 400 insoluble, typically solid, humins are removed from the stream 411a and disposed of via 413. The product is removed from the filter 400 via 414. The unconverted fructose stream 407b (and catalyst recovered from the liquid-liquid separation) is mixed with recovered solvent from stream 410 to form stream 410a which is resupplied to the mixer 100.

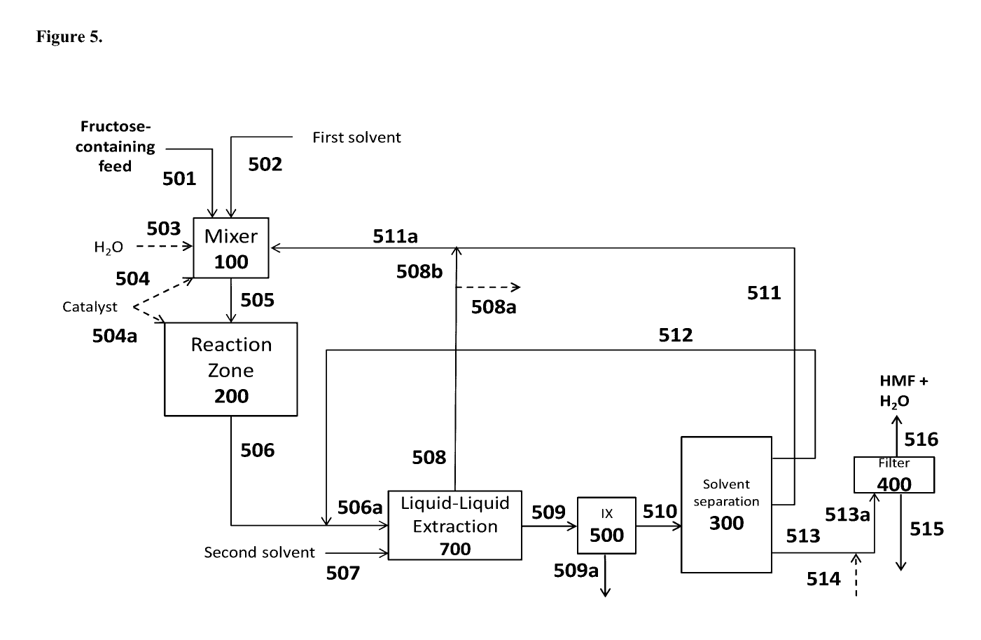

FIG. 5 illustrates a preferred embodiment of the partial conversion process of the present invention using a homogeneous catalyst and employing two solvents, one of which is employed to provide enhanced partitioning in fructose separator 700 for separating unconverted fructose from the combination removed from the reaction zone, for example, by employing liquid-liquid extraction technology. The configuration of major aspects of the process illustrated in FIG. 5 is the same as illustrated in FIG. 4. In this embodiment, an aqueous stream of fructose-containing feedstock is supplied via 501 to mixer 100 for mixing reaction constituents (e.g., a stirred tank). Also provided to mixer 100 via 502 is fresh and make up solvent, water provided via 503, and catalyst via 504. In this embodiment, catalyst may also be provided to a reaction zone 200 via 504a. Supply of catalyst to mixer 100 and reaction zone 200 need not be exclusive to either; instead, it may be supplied to both. The mixed reaction constituents are supplied to the reaction zone via 505. In the reaction zone 200, fructose is converted to HMF until the partial conversion endpoint is attained and then the conversion reaction is suitably quenched as described above. At least a portion of the reaction constituents, product (HMF and water), intermediates to HMF, solvent (in this embodiment the solvent may be polar or non-polar, preferably polar) and off-path products (such as levulinic acid, formic acid, and soluble and insoluble humins, among others) are removed from the reaction zone in combination and supplied via 506 to fructose separator 700 for separating unconverted fructose from the combination.

In one embodiment, fructose separator 700 is a liquid-liquid extraction apparatus. In this embodiment, a second solvent is added via 507 to the extractor 700. It is known to those skilled in the art that addition of a second solvent will affect the partition coefficient of the soluble components. The partitioned unconverted fructose and separated catalyst is removed via 508 and recycled to the mixer 100 as described in more detail hereinafter. A part of the partitioned unconverted fructose may optionally be purged via 508a as described above with respect to FIG. 4. Ultimately, not more than about 10%, typically not more than about 5%, or not more than about 2% of the unconverted fructose is contained in the liquid fed via 509 to catalyst recovery unit 500.

The remaining constituents partitioned into the layer that is the stream 509 (comprising product, catalyst, most or all of both solvents and off-path products) are delivered to catalyst recovery unit 500 (e.g., an ion exchange unit) designed, for example when HCl or H₂SO₄ is the catalyst, to capture the residual chloride or sulfate ions on the exchange resin prior to further processing steps. The “catalyst free” eluent from the ion exchange unit 500 is supplied via 510 to the solvent separator 300 for separating the solvents from the remaining constituents of the combination. In this embodiment, a distillation unit is utilized wherein fractions composed substantially of the first solvent and some water, preferably essentially only the first solvent, a fraction composed substantially of the second solvent and some water, preferably essentially only the second solvent, and a bottoms fraction comprised of product and off-path product can be withdrawn at appropriate, different locations along the length of the column. As illustrated in FIG. 5, separated first solvent is supplied via 511 as a component of the recycled mixture provided to the mixer 100 via 511a. Separated second solvent is recovered via 512 and supplied to the fructose separator 700 as, for example, a component of stream 506a (as shown) or directly to fructose separator 700 (not illustrated). The remaining product and off-path products withdrawn from solvent separator 300 via 513 are delivered via 513a, optionally with additional water supplied via 514, to filter 400. In filter 400 insoluble humins and other off-path products are removed from the stream 513a and disposed of via 515. The product is removed from the filter 400 via 516. The unconverted fructose stream 508b (and recovered catalyst) is then mixed with recovered first solvent stream 511 to form stream 511a which is resupplied to the mixer 100.

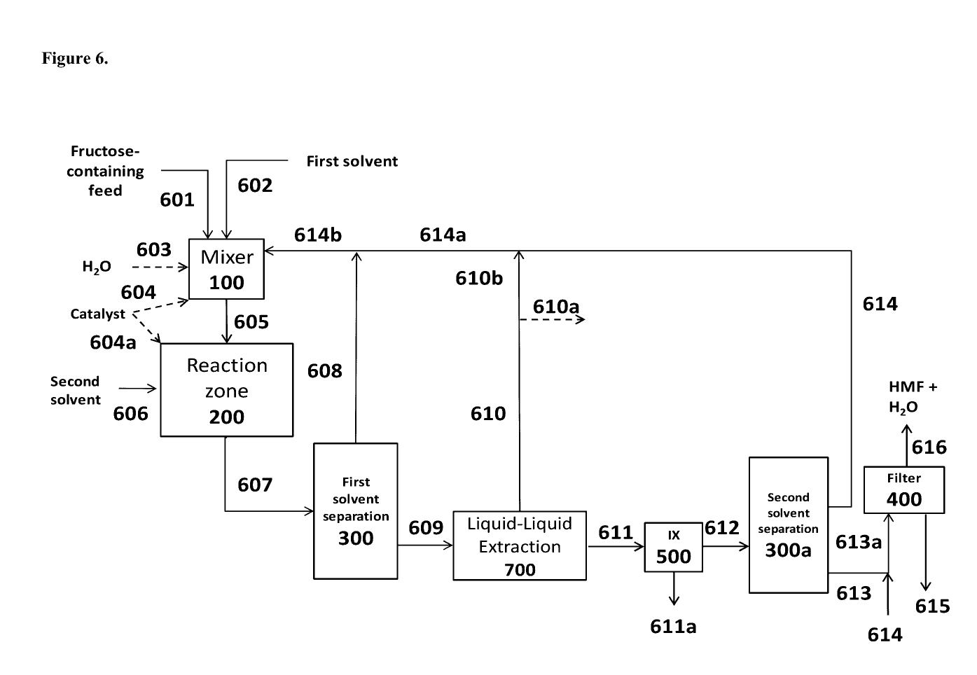

FIG. 6 illustrates an embodiment of the partial conversion process of the present invention using a homogeneous catalyst and employing two solvents, wherein both solvents are supplied to the reaction zone. In this embodiment, the configuration of major aspects of the process is different from that which is illustrated in FIG. 5 in that two solvent separators 300 and 300a are provided wherein one solvent separator 300 is provided upstream of fructose separator 700 to separate the first solvent from the combination removed from the reaction zone via 607 and another solvent separator 300a (which may be the same, similar to or different from solvent separator 300) provided downstream of fructose separator 700. In this embodiment, an aqueous stream of fructose-containing feedstock is supplied via 601 to mixer 100 for mixing reaction constituents (e.g., a stirred tank). Also provided to mixer 100 via 602 is fresh and make up first solvent, water provided via 603, and catalyst via 604. In this embodiment, catalyst may also be provided to a reaction zone 200 via 604a. Fresh and make-up second solvent is supplied to the reaction zone via 606. Although not illustrated, it will be apparent to those skilled in the art that the second solvent could be provided to the mixer 100. Supply of catalyst to mixer 100 and reaction zone 200 need not be exclusive to either; instead, it may be supplied to both. The mixed reaction constituents are supplied to the reaction zone via 605. In the reaction zone 200, fructose is converted to HMF until the partial conversion endpoint is attained and then the conversion reaction is suitably quenched as described above. At least a portion of the reaction constituents, product (HMF and water), intermediates to HMF, solvent (in this embodiment the solvent may be polar or non-polar, preferably polar) and off-path products (such as levulinic acid, formic acid, and soluble and insoluble humins, among others) are removed from the reaction zone in combination and supplied via 607 to solvent separator 300 for separating at least a portion of the first solvent from the combination removed from the reaction zone. The separated first solvent is removed via 608 to be resupplied to the mixer 100 as a component of stream 614b. The remainder from the solvent separator 300 is removed via 609 and supplied to fructose separator 700 for separating unconverted fructose from the combination removed from the reaction zone.

In one embodiment, fructose separator 700 is a liquid-liquid extraction apparatus. In this embodiment, the partitioned unconverted fructose (and catalyst) is removed via 610 and recycled to the mixer 100 as described in more detail hereinafter. Optionally, a purge may be affected via 610a to remove a portion of the unconverted fructose for any of a variety of reasons. Also, for example, means may be provided (not illustrated) to remove, for example, by another separation means (such as for example evaporation), a portion of the water that may have been partitioned with the unconverted fructose. Ultimately, not more than about 10%, typically not more than about 5%, or not more than about 2% of the unconverted fructose is contained in the liquid fed via 611 to catalyst recovery unit 500.

The remaining constituents partitioned into the layer that is stream 611 (in this embodiment product, residual catalyst, the second solvent and off-path products) are delivered to catalyst recovery unit 500 (e.g., an ion exchange unit) designed, for example when HCl or H₂SO₄ is the catalyst, to capture the residual chloride or sulfate ions on the exchange resin prior to further processing steps. The “catalyst free” eluent from the ion exchange unit 500 is supplied via 612 to solvent separator 300a for separating the second solvent from the remaining constituents of the combination. In this embodiment, a distillation or evaporation unit may be utilized depending upon the boiling point of the second solvent relative to that of the product wherein a fraction composed substantially of the second solvent and some water, preferably essentially only the second solvent, is removed via 614 and recycled to mixer 100 as a component of the constituents supplied via 614a and 614b to the mixer 100. The remaining product and off-path products withdrawn from the solvent separator 300a via means 613 are delivered, optionally with additional water supplied via 613a, to filter 400. In filter 400 insoluble humins are removed from the filter 400 as a stream 615 which may be disposed. The product is removed from the filter 400 via 616. The unconverted fructose containing stream 610b (and separated catalyst) is mixed with recovered second solvent and supplied via 614a to mix with recovered first solvent containing stream 608 to form stream 614b which is resupplied to mixer 100.

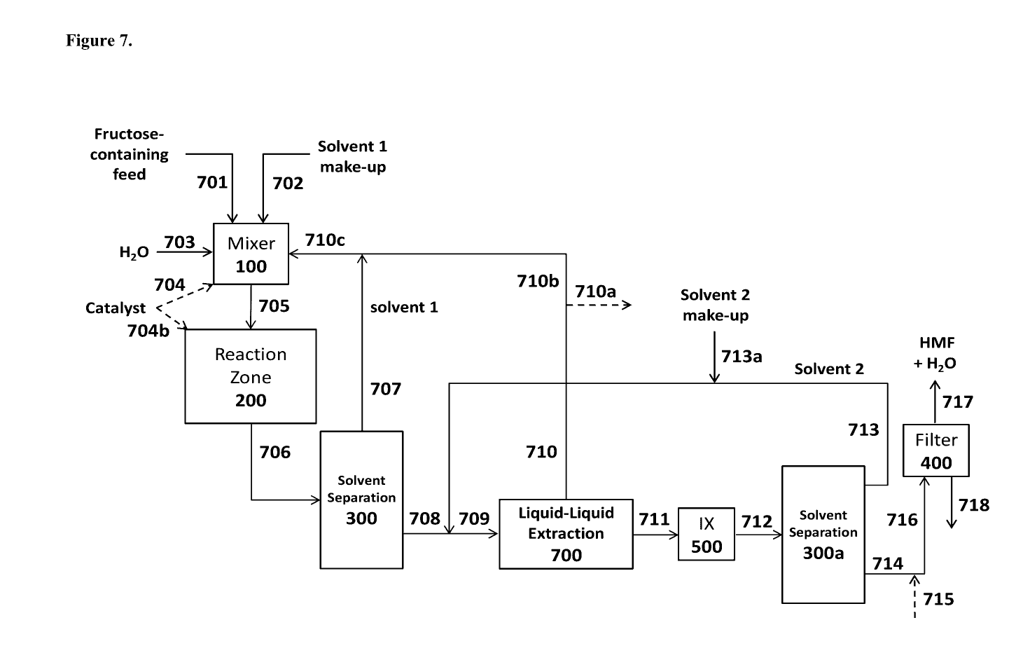

FIG. 7 illustrates another preferred embodiment of the partial conversion process of the present invention using a homogeneous catalyst and employing two solvents, one of which is employed to provide enhanced partitioning in fructose separator 700 for separating unconverted fructose, catalyst and intermediates from the product. In this embodiment, an aqueous stream of fructose-containing feedstock is supplied via 701 to mixer 100 for mixing reaction constituents (e.g., a stirred tank). Also provided to mixer 100 via 702 is fresh and make up first solvent. Water is provided via 703 and catalyst is supplied via 704 and/or 704b. The mixed reaction constituents are supplied to the reaction zone via 705. In the reaction zone 200, fructose is converted to HMF until the partial conversion endpoint is attained and then the conversion reaction is suitably quenched as described above. At least a portion of the reaction constituents, product (HMF and water), intermediates to HMF, solvent (in this embodiment the solvent may be polar or non-polar, preferably polar) and off-path products (such as levulinic acid, formic acid, and soluble and insoluble humins, among others) are removed from the reaction zone in combination and supplied via 706 to solvent separator 300 for separating at least a portion (preferably, substantially all) of the first solvent from the reaction combination. The solvent separation technique employed may be selected from among many options known to those skilled in the art (e.g., flash evaporation). The first solvent is removed as stream 707 for resupply to mixer 100 as a component of stream 710c.

The remaining constituents are removed from the first solvent separator 300 as stream 708. A second solvent, which is different from the first solvent, is added to stream 708 via 713. For example, in this embodiment, the first solvent can be an ether, such as DME and the second solvent can be a ketone, such as MIBK. The resulting stream 709 is supplied to fructose separator 700. Fructose separator 700 is a liquid-liquid extraction apparatus and separates a liquid phase comprising unconverted fructose, intermediates and catalyst from the composition of the stream 709. The partitioned liquid phase comprising unconverted fructose, intermediates and separated catalyst is removed via 710 and recycled to mixer 100 as described in more detail hereinafter. Optionally, a part of the liquid for any of a variety of reasons may be purged via 710a. For example, means may be provided (not illustrated) to remove, for example, by another separation means (such as for example evaporation), a portion of the water that may have been partitioned with the unconverted fructose.

The remaining constituents partitioned into the layer that is the stream 711 (comprising product, some catalyst, preferably substantially all of the second solvent and off-path products) are delivered to catalyst recovery unit 500 (e.g., an ion exchange unit) designed, for example when HCl or H₂SO₄ is the catalyst, to capture the residual chloride or sulfate ions on the exchange resin prior to further processing to recover product. Ultimately, not more than about 10%, typically not more than about 5%, or not more than about 2% of the unconverted fructose is contained in the liquid fed via 711 to the ion exchange unit 500. Upon effecting ion exchange to capture substantially all of the remaining catalyst, the “catalyst free” eluent from the ion exchange unit 500 is supplied via 712 to a second solvent separator 300a for separating the second solvent from the product. In this embodiment, a flash evaporation unit may be utilized to vaporize the second solvent and some water, preferably essentially only the second solvent. The bottoms fraction, now comprised of product and off-path materials can be withdrawn via 714. As illustrated in FIG. 7, separated first solvent from solvent separator 300 is supplied via 710b as a component of the recycled mixture provided to mixer 100 via 710c. Separated second solvent from second solvent separator 300a is recovered via 713 and resupplied to the fructose separator 700. Make-up second solvent, if needed, may be added via 713a. The remaining product and off-path materials withdrawn from second solvent separator 300a via 714 are delivered via 716, optionally with additional water supplied via 715, to filter 400. In filter 400 insoluble humins and other off-path materials are removed and disposed of via 718. The product is then removed from the filtration unit 400 as stream 717. The unconverted fructose containing stream 710b (and separated catalyst) is then mixed with recovered first solvent stream 707 to form stream 710c which is resupplied to mixer 100.

In another aspect of the invention, selective membrane separation techniques (e.g., ultra-filtration and/or nano-filtration) are employed to separate unconverted fructose, intermediates and HMF from the other constituents of the combination withdrawn from reaction zone. Selective membrane separation techniques utilized to treat the aqueous combination withdrawn from the reaction zone as disclosed herein provide effective recovery of unconverted fructose and intermediates for recycle, increased overall process yields and a high degree of product recovery.

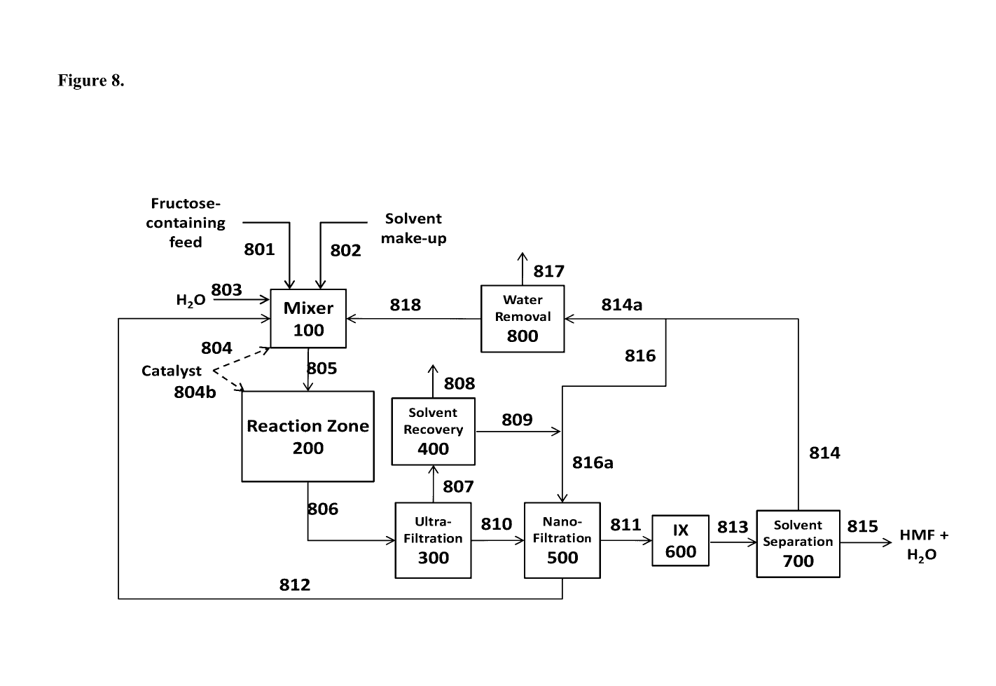

FIG. 8 illustrates another embodiment of the partial conversion process of the present invention using a homogeneous catalyst and an employing ultra-filtration unit 300 for the removal of humins, and a nano-filtration unit 500 for the separation of unconverted fructose and intermediates from the desired HMF product to enable the recycling of certain reaction constituents back to the reaction zone 200.

An aqueous stream of fructose-containing feedstock is supplied via 801 to mixer 100 for mixing reaction constituents (e.g., a stirred tank). Also provided to mixer 100 via 802 is fresh and make up solvent. Water is optionally provided via 803 and catalyst is supplied via 804 and/or 804b. The mixed reaction constituents are supplied to the reaction zone 200 via 805. In the reaction zone 200, fructose and reaction intermediates are converted to HMF until the partial conversion endpoint is attained and then the conversion reaction is suitably quenched as described above. At least a portion of the reaction constituents, product (HMF and water), intermediates to HMF, solvent (in this embodiment the solvent may be polar or non-polar, preferably polar) and off-path products (such as levulinic acid, formic acid, and soluble and insoluble humins, among others) are removed from the reaction zone in combination via 806 and subjected to selective membrane separation treatment as described in detail below.

The aqueous combination removed from the reaction zone intended for selective membrane separation treatment may be collected in an optional feed tank (not shown). In order to prevent fouling and the resulting loss of flux and extend the useful life of the selective membrane(s) employed in membrane separation unit(s), the suspended solids content in the aqueous combination removed from the reaction zone is optionally controlled. Typically, the aqueous combination will contain less than about 10,000 ppm of suspended solids. To enhance membrane performance and extend membrane life, the suspended solids content of the aqueous combination subjected to membrane separation may be reduced to less than about 1000 ppm, less than about 500 ppm, or less than about 100 ppm. The solids content of the aqueous combination removed from the reaction zone in 806 can be reduced, as necessary, to the desired level in an optional solids reduction stage (not shown). The solids reduction stage may represent a point of dilution wherein the aqueous combination is diluted with a quantity of an aqueous diluent (e.g., process water). Alternatively, the solids content of the aqueous combination can be reduced by a conventional filtration operation. The filtration operation can be suitably conducted in a batch mode (e.g., using bag filters) or in a continuous mode allowing for continuous flow of the aqueous combination through the solids reduction stage. Suitable continuous filters include cross-flow filters and continuous back-pulse filters wherein a portion of the filtrate is used to periodically back-pulse the filter media to dislodge and remove separated solids. Typically, the filter media employed is capable of separating and removing suspended solids greater than about 250 μm in size from the aqueous combination. It should be understood that any optional solids reduction stage may comprise a combination of dilution, filtration and/or other operations to attain the desired solids content in the aqueous combination prior to selective membrane separation treatment. The suspended solids content of the aqueous combination removed from the reaction zone can be readily determined by analytical methods known in the art such as by turbidity measurement (e.g., nephelometric turbidity units or NTU) and correlation of the turbidity reading to a known standard or by other methods known to those skilled in the art.

Following optional suspended solids reduction, the aqueous reaction combination withdrawn from the reaction zone is supplied via 806 to ultra-filtration unit 300 in which the aqueous reaction combination is contacted with one or more ultra-filtration membranes to produce a concentrate or retentate stream 807 containing at least a portion (preferably, substantially all) of the humins from the reaction combination and a permeate stream 810 containing unconverted fructose, intermediates, catalyst and HMF and depleted in humins relative to the aqueous reaction combination. Stream 807 is then fed to a solvent recovery unit 400 for the recovery of solvent from the humins-containing retentate stream. The humins are isolated via stream 808 and the recovered solvent stream 809 may be combined with stream 816 and supplied as diluents stream 816a to the downstream nano-filtration unit 500 as described below.

The ultra-filtration permeate stream 810 in combination with diluent stream 816a is supplied to nano-filtration unit 500 and contacted with one or more nano-filtration membranes to produce a permeate stream 811 containing HMF product, solvent and water and a retentate stream 812 containing at least a portion (preferably, substantially all) of the unconverted fructose and intermediates. Nano-filtration retentate stream 812 may also contain some portion of HMF and catalyst (i.e., homogeneous catalyst, if present) that did not permeate the nano-filtration unit 500. Nano-filtration permeate stream 811 may also contain catalyst, and some residual amounts of humins, fructose and reaction intermediates that have passed through the ultra-filtration and nano-filtration units. Stream 812 is supplied to mixer 100 for recycle to reaction zone 200.

The ultra-filtration unit 300 and nano-filtration unit 500 may comprise one or more ultra-filtration or nano-filtration membranes or modules and may be configured as either a single pass or multi-pass system, typically in a cross-flow arrangement wherein the feed flow is generally tangential across the surface of the membrane. The membrane modules may be of various geometries and include flat (plate), tubular, capillary or spiral-wound membrane elements and the membranes may be of mono- or multilayer construction. In some embodiments, tubular membrane modules may allow for higher solids content in the mother liquor solution to be treated such that solids reduction upstream of the membrane separation unit is not required or can be significantly reduced. The separation membranes and other components (e.g., support structure) of the membrane modules are preferably constructed to adequately withstand the conditions prevailing in the feed mixture and the membrane separation unit. For example, the separation membranes are typically constructed of organic polymers such as crosslinked aromatic polyamides in the form of one or more thin film composites. Specific examples of suitable ultra-filtration membranes include, for example and without limitation, spiral wound GE UF membranes having a molecular weight cut-off (MWCO) of 1000 available from GE Water & Process Technologies, Inc. (Trevose, Pa.), a division of GE Power & Water. Specific examples of suitable nano-filtration membranes include, for example and without limitation, spiral wound Dairy NF membranes having a MWCO of 150 and spiral wound H series membranes having a MWCO of 150-300 available from GE Water & Process Technologies, Inc.

Selective membrane separation techniques such as ultra-filtration and nano-filtration are pressure-driven separation processes driven by the difference between the operating pressure and the osmotic pressure of the solution on the feed or retentate side of a membrane. The operating pressure within a membrane separation unit will vary depending upon the type of membrane employed, as osmotic pressure is dependent upon the level of transmission of solutes through the membrane. Operating pressures in the membrane separation unit are suitably achieved by passing the feed stream (e.g., incoming reaction constituents in the combination removed from the reaction zone) through one or more pumps upstream of the membrane unit, for example, a combination booster pump and high-pressure pump arrangement. Generally, ultra-filtration operations exhibit lower osmotic pressures than nano-filtration operations, given the same feed solution. The driving force for transmission through the membrane (i.e., permeate flux) increases with the operating pressure. However, the benefits of increased operating pressure must be weighed against the increased energy (i.e., pumping) requirements and the detrimental effects (i.e., compaction) on membrane life.

Typically, the operating pressure utilized in the ultra-filtration operation is less than about 800 kPa absolute and preferably from about 200 to about 500 kPa absolute. Typically, the operating pressure utilized in the nano-filtration operation is less than about 1200 kPa absolute and preferably from about 600 to about 900 kPa absolute. High temperatures tend to decrease the useful life of selective membranes. Accordingly, the temperature of the aqueous combination introduced into the ultra-filtration membrane separation unit 300 is generally from about 20° C. to about 100° C., and typically from about 30° C. to about 60° C. or from about 30° C. to about 50° C. If necessary, the aqueous combination can be cooled prior to being introduced into membrane separation unit 300 by methods conventionally known in the art including, for example, indirect heat exchange with other process streams or with cooling water (e.g., as part of the quench step).

In order to maintain or enhance membrane separation efficiency and permeate flux, the membranes should be periodically cleaned so as to remove contaminants from the surface of the membrane. Suitable cleaning includes cleaning-in-place (CIP) operations wherein the surface of the membrane is exposed to a cleaning solution while installed within ultra-filtration unit 300 and nano-filtration unit 500. Some systems monitor the conductivity of the permeate, as conductivity can be correlated to the concentration of components that pass through the membrane. An increase in conductivity in the permeate may indicate an increase in transmission of the desired retentate compounds through the membrane and can be used to signal the need for cleaning operations. Additionally, a fall in permeate flow with all other factors remaining constant may indicate fouling and the need for cleaning operations. Cleaning protocols and cleaning solutions will vary depending on the type of separation membrane employed and are generally available from the membrane manufacturer. In order to not damage the membranes and unnecessarily shorten membrane life, the CIP operation is preferably conducted using a solution of a standard pH at pressure and temperature conditions known to those skilled in the art. In some applications, it may be advantageous to conduct a cleaning operation on new separation membranes prior to use in the membrane separation operation in order to improve membrane performance.

The nano-filtration permeate stream 811 is delivered to an optional catalyst recovery unit 600. For example, catalyst recovery unit 600 may comprise an ion exchange unit designed, for example when HCl or H₂SO₄ is the catalyst, to capture the residual chloride or sulfate ions on the exchange resin prior to further processing to recover the HMF product. Ultimately, not more than about 10%, and typically not more than about 5%, or not more than about 1% of the unconverted fructose and reaction intermediates are contained in the liquid fed via 811 to the ion exchange unit 600. Upon effecting ion exchange to capture substantially all of the remaining catalyst, the “catalyst free” eluent from the ion exchange unit 600 is supplied via 813 to a solvent separator 700 for separating the solvent and a portion of the water from the product. For example, a flash evaporation unit may be utilized to vaporize the solvent and some water, preferably essentially only the solvent. The bottoms fraction, now comprised of primarily HMF and water can be withdrawn via 815.

Separated solvent from solvent separator 700 is recovered in 814. Stream 814 optionally provides diluent for nano-filtration unit 500 via 816. The remainder of the stream is supplied to the water removal unit 800 via 814a. A portion (preferably, substantially all) of the water in stream 814a can be removed as stream 817 employing of a number of methods including, but not limited to, distillation, adsorption, pervaporation and membrane separation. The water-reduced stream 818 containing primarily solvent is supplied to mixer 100 for recycle to reaction zone 200.

The process described by FIG. 8 contains solvent separator unit 700 which can be used to remove solvent and produce stream 815 containing HMF and water. In an alternative embodiment, unit 700 may configured to remove water via stream 814 (either as a pure water stream or as an azeotrope with the solvent) producing stream 815 containing HMF and solvent, which may optionally contain some water.

While the various process schemes illustrated in the accompanying Figures provide for a product containing HMF as an aqueous solution, it will be evident to one of skill in the art that any of the process schemes may be readily adapted to produce HMF dissolved in a solvent other than water, or HMF dissolved in a solvent/water combination.

In Situ Reduction of HMF to BHMF

Another aspect of the invention is the production of HMF by way of an in situ transformation of HMF to the intermediate 2,5-bis-hydroxymethylfuran (BHMF). Accordingly the present invention provides, a process for the production of 2,5-bis-hydroxymethylfuran (BHMF) and/or 2,5-bis-hydroxymethyltetrahydrofuran (BHMTHF), the process comprising: combining a sugar, an acid catalyst, a hydrogenation catalyst, and a solvent under conditions suitable for converting the sugar to BHMF and/or 2,5-bis-hydroxymethyltetrahydrofuran (BHMTHF). BHMF is more stable than HMF and is less susceptible to subsequent reactions that form humins. See, Example 12. BHMF may be further converted to the intermediate 2,5-bis-hydroxymethyltetrahydrofuran (“BHMTHF”), which is also less susceptible to humins-forming reactions.

An important aspect of this process is that HMF can be produced via the dehydration of fructose and then subsequently reduced to form BHMF. As HMF is formed, the concentration remains low, thereby limiting the rate of humins formation. The yield of BHMF should be at least about 80%, in various embodiments at least about 85%, more typically at least about 90%, and in various preferred embodiments at least about 95%. BHMF is highly useful industrially as it can be used as an intermediate for the high yielding production of 1,6-hexanediol, hexamethylenediamine, BHMTHF, 1,2,6-hexanetriol, caprolactone, and the like. Exemplary conditions for converting the sugar to BHMF include the temperature and pressure ranges described herein above for the partial conversion of fructose to HMF.

The solvent used in this process may be water, an organic solvent or a mixture of water and an organic solvent that is either miscible or immiscible in water. With an immiscible organic solvent, the hydrogenation can take place in the organic solvent and the dehydration can take place in water with the resultant HMF partitioning between the water and the organic solvent. Such an embodiment allows for the separation of reaction conditions for the dehydration and the hydrogenation steps, which is advantageous if the dehydration and reduction conditions are not compatible. Alternatively, the hydrogenation may take place in the aqueous phase and the dehydration may take place in the organic solvent.

In one aspect of this process, the rate of dehydration is preferably slower than the rate of hydrogenation. As will be appreciated by a person of ordinary skill in the art, reaction conditions can be adjusted to control the rate of dehydration. For example temperature, solvent composition, catalyst and catalyst loading can all be tailored to a desired dehydration rate that is equal to or less than the rate of the hydrogenation. In some preferred aspects of the invention, selectivity of the fructose to HMF reaction is greater than about 85%, or preferably greater than 90%, in some embodiments, greater than 95% or 97%.

In one aspect, a hydrogenation catalyst can be used to selectively reduce the aldehyde group of HMF to form BHMF in high yield. Suitable hydrogenation catalysts include heterogeneous catalysts, for example, catalysts which comprise (1) at least one metal from the group consisting of Ni, Co, Cu, Ag, Au, Pt, Pd, Fe, Rh, Ir and Ru, and mixtures thereof, and (2) a support such as acid-functionalized resins, acidified carbons, zeolites, micro- and meso-porous metal oxides, sulfonated and phosphonated metal oxides, clays, polyoxometallates and combinations thereof. In some embodiments, the heterogeneous catalysts include acid functionalized resins. The acid catalyst may be a homogeneous acid catalyst as described herein above or a heterogeneous (i.e., solid) acid catalyst as illustrated in Example 12). Modifiers or promoters may be used to tune the selectivity for the reduction of the aldehyde group of HMF over the keto-group of fructose. Reduction of the keto-group present in fructose leads to the formation of mannitol or sorbitol which is not amenable to dehydration to HMF. Suitable modifiers and/or promoters include Au, W, Cu, Zn, Mo, Bi, Sb and Pb.

A homogeneous reduction catalyst may also be used, for example a catalyst comprising at least one metal from the group consisting of Ni, Co, Cu, Ag, Au, Pt, Pd, Fe, Rh, Ir and Ru. Ligands which bind to the metal may also be used to tune the selectivity for the reduction of the aldehyde group of HMF over the keto-group of fructose.

Where the catalyst for the dehydration is a mineral acid in a single solvent or a single-phase solvent mixture, the reduction catalyst can be selected to be compatible with the mineral acid and may be selected from the group consisting of Ag, Au, Pt, Pd, Ru Rh and Ir. Where the catalyst for the dehydration is a mineral acid in a 2-phase solvent mixture, the reduction catalyst may be selected from the group consisting of Ni, Co, Cu, Ag, Au, Pt, Pd, Fe, Rh, Ir and Ru.

The temperature of the reduction step may be the same temperature used for the dehydration step. The rate of hydrogenation may be equal to or greater than the rate of dehydration. The reactions may be tailored by modifying conditions such as metal selection, metal loading, catalyst loading hydrogen pressure and solvent choice. A hydrogen pressure between 50 and 1000 psi may be used. In other embodiments, the hydrogen pressure is greater than 1000 psi, for example, about 1100 psi, or about 1150 psi, or about 1200 psi, or about 1400 psi, or about 1600 psi, or about 1800 psi, or about 2000 psi.

In some aspects of the invention, the selectivity of the HMF to BHMF reaction is greater than about 95% or about 97%, preferably greater than about 98% or about 99%. In some aspects of the invention, there is a high selectivity for BHMTHF formation, for example, the selectivity can be greater than about 95% or about 97%, preferably greater than about 98% or about 99%.

In a further embodiment, the source of hydrogen for the reduction step is any reagent known in the art to be suitable for transfer hydrogen. For example the hydrogen transfer reagent may include a secondary alcohol resulting in hydrogen transfer from the alcohol to HMF with the subsequent formation of BHMF and a ketone, which may be separated, reduced back to the secondary alcohol and recycled for further use. See for example Chem. Cat. Chem. 2014, Issue 2, Volume 6, pages 508-513. Other hydrogenation transfer reagents, catalysts and conditions are also known in the art.

HMF Functional Group Protection

Another aspect of the invention is a method of preserving HMF by way of functional group protection methods. HMF contains a highly reactive aldehyde group that is vulnerable to further reaction under aggressive reaction conditions. For example, at high temperatures in the presence of an acid catalyst to form HMF from fructose, further reaction of the HMF aldehyde group can occur (for example, acid catalyst condensation reactions with fructose, dehydration intermediates or condensation of HMF itself) to produce oligomeric condensation products such as humins. In one aspect of the invention, high yields of HMF may be achieved by functional group protection of the aldehyde group, which prevents condensation reactions as HMF is formed during the dehydration reaction. This allows more complete conversion of fructose to aldehyde-protected HMF without the formation of humins. The aldehyde-protected HMF can then be deprotected to form HMF in high yield.

Suitable aldehyde protecting groups include, but are not limited to, cyclic and acyclic acetals, thio- and dithio-cyclic and acyclic acetals, seleno- and diseleno-cyclic and acyclic acetals, cyanohydrins, hydrazones, oximes, 1,2-adducts with cyclic and acyclic amine adducts, reversible formation of enolate anions, enol ethers, enamines and imines.

In some aspects this process requires a) selective protection of the aldehyde of HMF in the presence of the keto-group of fructose and b) a protecting group that is stable under the conditions of fructose dehydration, and c) high yielding removal of the protecting group and recovery of the HMF and the protecting group.

Functional group protection strategies are known in the art. See, for example, Greene and Wuts, Protective Groups in Organic Synthesis, Wiley and Sons 2007. The functional group method of the invention advantageously prevents the reaction or decomposition of the HMF during reaction conditions. The protected functional group may therefore be exposed to reaction conditions that would otherwise cause the functional group to react or decompose. The functional group can therefore be preserved during incompatible reaction conditions and recovered after the reaction by deprotection, i.e. removal of the protective group.

Modulating Isomeric Forms of Fructose

Another aspect of the invention relates to the synthesis of HMF using advantageous isomeric forms of fructose. See, Examples 13 and 14. Without being bound by any theory, it is noted that fructose can exist in several different isomeric forms that are in equilibrium with each other. In aqueous solution, for example, fructose can exist in a linear acyclic form that can interconvert to either cyclic pyranose forms, or cyclic furnanose forms. Both of the cyclic forms are hemiketal isomers of the linear acyclic form of fructose and can interconvert via equilibrium with the linear acyclic form. The hemiketals can exist in either α- or β-anomeric forms and so four possible tautomeric cyclic structures can exist in solution. It is believed that at equilibrium in water at ambient temperatures the dominant form of fructose is the β-pyranose form. β-furanose is believed to be the second most abundant form, with lower levels of the α-furanose and the acyclic forms also being present. The equilibrium mixture of the composition and the kinetic rate to form an equilibrium of the various fructose forms is believed to be dependent on the nature of the solvent composition (polarity, ionic strength) and temperature. It is also believed that the 5-membered ring structures of the fructose furanose forms exhibit the highest propensity to dehydrate and form HMF. Therefore the use of appropriate conditions to favor the formation of the furanose forms of fructose can provide high HMF yields. The pyranose form of fructose can, under conditions favorable to dehydration, lead to the formation of unwanted side products. See Horvath in Chem. Commun. 2012, Vol. 48, p. 5850. Solvent and temperature effects on the equilibrium concentrations of the furanose and pyranose forms have also been reported. See for example Schallenberger in Pure & Applied Chem. Vol. 50, p. 1409, Goux in J. Am. Chem. Soc. 1985, Vol. 107, p. 4320 and Matubayasi in J. Phys. Chem. A. 2013, Vol. 117, p. 2102.

In an important aspect of the invention, HMF reaction conditions that favor furanose forms of the reactant can provide facile production of HMF at high yields, of about 85%, or preferably about 90%, or in some cases about 95% or about 97% HMF.

In certain embodiments, an appropriate solvent or solvent composition is used to convert fructose to HMF in two stages. Stage 1, for example, is a pre-equilibrium stage in which fructose is dissolved in a suitable solvent composition and is held at an appropriate temperature (optionally including a catalyst, which may be a Lewis acid or a Bronsted acid catalyst which may be a homogeneous or a heterogeneous catalyst) for an appropriate time to enable the preferential formation of the furanose form of fructose. The pre-equilibrium stage may also enable dehydration reactions of the furanose form of fructose to a certain extent. Dehydration reactions of the furanose form of fructose are on the reaction pathway to the formation of HMF and prevent reformation of the pyranose form of fructose. The temperature range of Stage 1 is from room temperature to 120° C. Suitable solvents may include, but are not limited to, water and water miscible organic solvents and combinations thereof.

Stage 2 of this process is a reaction stage in which the equilibrium mixture containing the furanose form of fructose (which may include the partially dehydrated furanose form of fructose) is converted to HMF. The catalyst may be a Lewis acid or a Bronsted acid catalyst which may be a homogeneous or a heterogeneous catalyst. The Stage 2 reaction stage can also include multiple temperature zones and/or multiple zones for the injection of additive (for example a solvent or a solvent composition). Multiple temperature zones or zones for the injection of additive may be helpful in preserving high selectivity for the formation of HMF during the reaction pathway.

The rate of formation of the equilibrium mixture of the fructose isomers can depend on the ionic strength of the solvent. The ionic strength of the solvent can be adjusted by the addition of ionic salts. In a further embodiment, an ionic salt can be added to the solvent or solvent composition. Additionally, it is believed that the ionic salt can influence the rate of formation of the equilibrium mixture which can enable more preferable conditions for Stage 1 (for example the favorable equilibrium mixture containing the furanose form of fructose as the dominant form may be readily accessed at lower temperatures which can limit unwanted side reactions thereby ensuring high selectivity for the conversion of the furanose form of fructose to HMF). Furthermore, since the dehydration of fructose to HMF is believed to involve intermediate ion pairs (for example, upon protonation of fructose), the ionic strength of the solvent or solvent combination is believed to advantageously influence the reaction. Preferred salts for use in this process include, but are not limited to, alkali metal salts, alkaline earth salts, ammonium salts or quaternary amine salts. Certain preferred salts may contain anions which may be non-coordinating anions (such as BF₄⁻, PF₆⁻, BPh₄, perfluoroarylborates, carboranes etc) or halide anions, or more preferably a bromide anion. Preferred salts include selected MBr or R₄₋ₓHₓNBr where M is Li, Na, K, Cs, Mg, Ca, Sr, Ba and each R is independently selected from linear or branched or cyclic hydrocarbyl group which may be an aliphatic or an aromatic hydrocarbyl group. Especially preferred salts include MBr or R₄₋ₓHₓNBr where M is Na, K, Cs, Mg, Ca, and each R is independently selected from a C₁-C₁₂ linear or branched or cyclic alkyl group. A preferred quaternary amine salt is tetramethylammonium bromide and tetraethylammonium bromide. Additionally, the quaternary ammonium salt may be a solid phase reagent, such as an anion-exchange resin containing a quaternary ammonium functional group in the halide form, or more preferably the bromide form. The ionic salt may be used in combination with any of the previous concepts described.

### Modulating Fructose Concentration

In one aspect of the invention, it has been found that the concentration of undesirable humins during HMF synthesis can be controlled by modulating the concentration of the reactants necessary for the formation of humins. In one such embodiment, the humins concentration is diminished using a low fructose concentration. Low fructose concentrations are generally not beneficial to industrial processes because of the expense required to remove the high concentrations of solvent necessary to isolate the product. However, an industrially viable method to keep fructose concentrations low has been discovered, by introducing fructose into the reaction zone in solid particle form.

Particles of fructose may be introduced into an appropriate reactor containing a solvent in which fructose is only soluble to a certain extent (for example <10 wt. % or preferably <5 wt. %). The solvent can contain a Lewis acid or a Bronsted acid catalyst (which may a homogeneous or a heterogeneous catalyst). In some embodiments of this aspect of the invention, particles of fructose only dissolve in the solvent to a certain extent, thereby keeping the concentration of fructose and reaction intermediates low and restricting formation of humins. This can produce high yield of HMF, for example about 95%, or about 97%, or about 99% or greater.

The solvent is chosen to solubilize the HMF formed from the reaction. In some embodiments, a low water concentration is used in combination with another solvent (for example less than about 10 wt. %, or less than about 5 wt. %), although the water concentration may increase during the reaction as a consequence of water formation from the dehydration of fructose. Any suitable method for feeding the fructose particles (e.g. screw feeders) may be used in conjunction with any suitable industrial reactor format (stirred tank, fluidized bed etc.), as will be appreciated by those of ordinary skill in the art.

Any of the previously described aspects of the invention may be used independently or together in a dehydration reaction for the conversion of fructose to HMF to afford high yields of the HMF product (for example greater than about 85%, preferably greater than about 90%, or greater than about 95%, more preferably greater than about 97%). The yields described may be obtained in a dehydration reaction in which fructose is fully converted, or in which the fructose is partially converted and the HMF is separated from the residual fructose (and reaction intermediates) by known separation methods. The residual fructose is either recycled to the dehydration reactor or converted through to produce more HMF by the use of a second dehydration reactor which can be the same or different from the first dehydration reactor and the condition used in the second dehydration reactor may be the same or different as the condition used in the first dehydration reactor.

### Organic Solvent-Compatible Membranes

In another aspect of the invention, it has been discovered that certain organic solvent-compatible membrane materials provide a surprising efficacy for separating HMF from humins and unconverted fructose using a solvent combination comprising water and a water miscible organic solvent. See, Examples 15-18. These membranes are compatible with water miscible organic solvents and do not impart detrimental impact on performance. The separations can be conducted at low pH or the reaction mixture can be neutralized prior to the membrane separation. The neutralization step can include addition of a base such as NaOH, CaCO₃ or Ca(OH)₂. The base can be selected such that the resultant salt is rejected by the membrane which enables the separation of humins, along with the salt resulting from the neutralization. Polymeric and ceramic membranes can be used.

In this aspect of the invention, membrane separation can be used for multiple process embodiments of the invention, for example, fructose can be fully converted through to the reaction product and an ultrafiltration ceramic or polymeric membrane can be used to separate humins from the HMF which is produced as a permeate stream. In this embodiment, the catalyst can be a Lewis acid or a Bronsted acid and may be a homogeneous or a heterogeneous acid. In the embodiment in which a homogeneous acid is deployed, it may optionally be neutralized and removed by the ultrafiltration membrane as a salt, thereby removing the catalyst from the HMF permeate stream.

In other aspects of the invention, fructose can be converted to a partial conversion endpoint and an ultrafiltration membrane may be first used to separate humins from the HMF and unconverted fructose and any reaction intermediates which are produced together as a permeate stream. In this embodiment, the catalyst can be a Lewis acid or a Bronsted acid and may be a homogeneous or a heterogeneous acid. In the embodiment in which a homogeneous acid is deployed, it may optionally be neutralized and removed by the ultrafiltration membrane as a salt, thereby removing the catalyst from the permeate stream containing the HMF, unconverted fructose and any reaction intermediates. The permeate stream may then be subjected to nanofiltration membrane separation in which the HMF is separated from the unconverted fructose and reaction intermediates. The HMF stream may be produced as a permeate stream from the nanofiltration separation and the retentate stream containing the unconverted fructose and any reaction intermediates can be either recycled to the dehydration reactor or converted through to more HMF by the use of a second dehydration reactor which can be the same or different from the first dehydration reactor. Additionally, the reaction conditions used in the second dehydration reactor may be the same or different as the reaction conditions used in the first dehydration reactor.

### Alumina and Carbon Adsorbents

In another aspect of the invention, a particular class of materials capable of selectively removing humins and certain other contaminants from the dehydration reaction product has been discovered. See, Examples 9-11. In certain aspects, for example, aluminas may be used to remove contaminants and are advantageously used to remove the dehydration catalyst directly from the dehydration reaction product optionally without the need for neutralization, membrane separation or without the need for ion-exchange removal. The alumina materials may be deployed anywhere downstream of a dehydration reactor where removal of humins or catalyst or certain other unwanted reaction products is desired. The aluminas can be regenerated after becoming saturated with an adsorbing species. Preferred regeneration methods are either chemical or thermal regeneration methods. Such regeneration protocols are known in the art and may be deployed with the frequency necessary to ensure that the alumina retains its capacity for the effective removal of humins or catalyst or certain other unwanted reaction products from the product stream. Such protocols are typically deployed using multiple fixed bed columns of alumina that are either deployed in operation to remove the humins catalyst or certain other unwanted reaction products from the product stream, or they are undergoing regeneration or washing protocols to restore adsorbent performance, or they are standing by in a state of readiness in anticipation of being brought into. In some aspects of the invention, certain carbon materials may be used as adsorbents for the removal of humins or certain other unwanted reaction products. In general, any type of carbon known for adsorption properties can be used. In particular, activated carbon is preferred. Suitable carbon materials include forms of elemental carbon, such as, for example, activated carbon, carbon black, graphite, carbon nanotubes, and the like. A further preference is an activated carbon that can be regenerated using chemical or thermal methods. A preferred regeneration method is a chemical regeneration method. The carbons can be deployed anywhere downstream of a dehydration reactor where removal of humins or certain other unwanted reaction products is desired. Regeneration protocols are known in the art and may be deployed with the frequency necessary to ensure efficacy of removal of humins and certain other unwanted reaction products from the product stream. Such protocols are typically deployed using multiple fixed bed columns of carbon that are either deployed in operation to remove the humins and certain other unwanted reaction products from the product stream, or they are undergoing regeneration or washing protocols to regenerate absorbent performance or they are preserved in a state of readiness in anticipation of being brought into operation to remove the humins and certain other unwanted reaction products from the product stream.

Alumina and carbons can be also used together (for instance in serial fixed beds) to optimize the adsorbent performance.

### Continuous Flow Reactor

In another aspect of the invention, a continuous flow dehydration reactor is provided in which fructose can be pumped though a dehydration reaction zone and selectively dehydrated to a partial conversion endpoint to produce HMF with very high selectivity with little or no humins. In some embodiments, the solvent comprises water and a water miscible organic solvent. The dehydration catalyst can include a homogeneous or a heterogeneous dehydration catalyst which can be a Lewis acid or a Bronsted acid catalyst.

The reaction zone can convert fructose to HMF at a partial conversion endpoint. The partial conversion endpoint can be controlled by the temperature of the reaction zone and the time the reaction mixture remains in contact with the reaction zone (residence time). The temperature of the reaction zone can be from 80° C.-200° C. or more, preferably from 100° C.-180° C. The residence time in the reactor is chosen to limit the fructose conversion. Preferably the fructose conversion is within the range 10-50% or more, or preferably within the range 10-35%. Having the partial conversion controlled within these ranges, in combination with the preferred reaction conditions, fructose can be converted to HMF with very high selectivity with little or no humins formation. The dehydration reactor is any type of continuous flow reactor or more preferably a continuous flow tubular reactor. With a continuous flow tubular reactor, the reactor length can be chosen to control the residence time in the reactor. A cascade of continuous flow tubular reactors can optionally be used to reduce overall reactor volumes. The reaction product from the outlet of the dehydration reactor can be fed into a membrane separation unit. The membrane separation unit can be used to allow HMF to permeate through the membrane and retain the unconverted fructose to be recycled in the dehydration reactor. In one embodiment, the membrane is a polymeric membrane. In a further embodiment, the polymeric membrane is a nanofiltration membrane. In a further embodiment the nanofiltration membrane is arranged in a spiral wound assembly which is used to enable a cross-flow filtration. In a further embodiment, the reaction product is fed through the spiral wound nanofiltration membrane assembly with a flow of ≧20 cms⁻¹. Additionally, the dehydration reactor can be designed such that the fructose can be fed through the continuous reactor at the same velocity of ≧20 cms⁻¹ in which the fructose conversion is controlled within the range of 10-50%, or more preferably within the range of 10-35%. In a further embodiment, the surface area of the membrane with the spiral wound assembly (or multiplexed assemblies) is chosen such that recovery of the HMF produced in the partial conversion reaction reaches a minimum of 85% (for example greater than about 85%, preferably greater than about 90%, or greater than about 95%, more preferably greater than about 97%) in the combined permeate streams. In a further embodiment, the retentate from the membrane filtration, which contains the unconverted fructose, and optionally the acid catalyst, is recycled back to the dehydration reactor. In a further embodiment, the retentate from the membrane filtration is subjected to a purge prior to its recycling to the dehydration reactor. The purge stream can serve to enable removal of unwanted components that could otherwise build up in the recycle loop from the reaction, such as low levels of unwanted reaction products such as humins, or unwanted components that may be present in the fructose feedstock (e.g glucose or disaccharides or oligosaccharides). The purge stream may optionally deploy an ultrafiltration membrane to remove the low levels of humins and recycle the permeate containing the unconverted fructose back to the dehydration reactor. The ultrafiltration membrane can be a polymer membrane or a ceramic membrane. The purge stream may also be passed over an absorbent to remove the low levels of humins and recycle the permeate containing the unconverted fructose back to the dehydration reactor. The absorbent may be an alumina or a carbon-based absorbent. The product of the reaction from this concept is the permeate stream from the nanofiltration unit which is a solution comprising water, a water miscible organic solvent and HMF. The purity of the HMF in this stream can be greater than or equal to about 90%, or about 95%, or about 97% or preferably about 99%. A continuous flow dehydration reactor and membrane separation unit in a connected loop with a optional purge stream can be used to produce a solution of HMF in which the purity of the HMF in the solution stream can be greater than or equal to about 90%, or about 95%, or about 97% or preferably about 99%.

All of the embodiments described herein that involve the dehydration of fructose to HMF may be preferentially carried out in the absence of oxygen or air, or in the present of an inert gas such as nitrogen in order to avoid any adverse reactions of oxygen with any of the reactants or product components.

Quantification of humins in a reaction sample may be performed by visual inspection of the sample color/UV analysis (increased humins, correlates qualitatively with darkness of the sample solution) and mass balance (e.g., in which all other components in the product mixture are quantified and the difference between the starting mass and the mass of the final product is determined).

### Conversion of Xylose to Furfural

In some aspects of the invention, a method for converting of xylose to furfural is provided. In this process a dehydration reaction in which xylose, a 5-carbon sugar, can be subjected to dehydration reaction conditions to produce furfural, an important intermediate that can be converted to a variety of 5-carbon chemicals of industrial importance. The dehydration reaction has many properties in common with the dehydration reaction of fructose to HMF. The reaction can be catalyzed by a Lewis acid or a Bronsted acid catalyst which can be a homogeneous or a heterogeneous acid catalyst. The dehydration of xylose to furfural is known to produce undesirable side products. In many instances the undesirable side product are also darkly colored oligomers or polymers also often referred to as humins. The humins are also thought to result from condensation reactions that can occur between furfural, xylose and reaction intermediates. The condensation reactions are also bimolecular reactions that lead to compounds of increased molecular weight along with the elimination of water. Consequently, the embodiments described above for the dehydration of fructose to HMF are also suitable for application in a process that dehydrates xylose to furfural.

As used herein, the term “undesired sugar dehydration by-product” refers to humins, a hydroxyacetylfuran (e.g., 2-hydroxyacetylfuran), and the like. Typically, the undesired sugar dehydration by-product is a humin.

### EXAMPLES

The following non-limiting examples are provided to further illustrate the present invention.

### Example 1

Fructose, water, HCl, NaCl and organic solvent were combined in a sealed reactor in the proportions detailed in Table 1. The reactor was heated with stirring to the temperature and for the time reported in Table 1. On cooling, samples of all layers were taken and the products were analyzed and composition determined by HPLC. HPLC analysis in Examples 1 through 6 was conducted on an Agilent 1200 LC system using a Thermo Scientific Hypercarb, 3.0×30 mm, 5 um column (guard) and an Agilent Zorbax SB-Aq 3.0×100 mm, 3.5 um column (analytical) at 46° C. The species were eluted under isocratic conditions of using a mixture of 90% (v/v) solvent mixture A (0.1% formic acid in water) and 10% (v/v) solvent mixture B (0.1% formic acid in 50:50 methanol:water) at a flow rate of 1.0 mL/min. Fructose, glucose and intermediates were detected using a universal charged aerosol detector (CAD), while HMF was detected by UV at 254 nm. Fructose, glucose and HMF were quantified by fitting to calibration curves generated from pure standards. Intermediates were quantified using a calibration curve generated from a structurally related compound. The distributions of products are described in Table 1.

TABLE 1

| | | | | | Total | | | | | | | Sum of mol % |
|---|---|---|---|---|---|---|---|---|---|---|---|---|
| | | | | | Solvent | | Run | Run | Unconverted | Inter- | | Fructose + |
| | Fructose | HCl | Water | | Added | NaCl | Temp. | Time | Fructose | mediates | HMF | Intermediates + |
| Entry | wt % | mol % | wt % | Solvent | (mL) | (mg) | (° C.) | (min) | mol % | mol % | mol % | HMF |
| 1 | 20 | 5 | 20 | 2Butanol | 4 | 130 | 120 | 30 | 35 | 16 | 49 | 100 |
| 2 | 20 | 5 | 20 | 2-Butanol | 4 | 0 | 140 | 15 | 29 | 20 | 47 | 96 |
| 3 | 10 | 5 | 15 | Diglyme | 4 | 0 | 100 | 60 | 26 | 26 | 43 | 95 |
| 4 | 10 | 10 | 15 | Diglyme | 4 | 0 | 100 | 30 | 26 | 26 | 43 | 94 |
| 5 | 10 | 20 | 15 | Diglyme | 4 | 0 | 100 | 15 | 30 | 21 | 43 | 94 |
| 6 | 10 | 10 | 20 | Diglyme | 4 | 0 | 100 | 60 | 36 | 20 | 41 | 97 |
| 7 | 10 | 10 | 20 | Diglyme | 4 | 0 | 100 | 60 | 37 | 20 | 42 | 99 |
| 8 | 10 | 15 | 20 | Diglyme | 4 | 0 | 100 | 30 | 33 | 24 | 41 | 99 |
| 9 | 10 | 5 | 20 | Dioxane | 2 | 0 | 130 | 15 | 35 | 19 | 46 | 100 |
| 10 | 10 | 5 | 20 | Dioxane | 2 | 0 | 140 | 15 | 45 | 13 | 41 | 100 |
| 11 | 15 | 10 | 15 | Glyme | 4 | 0 | 110 | 30 | 26 | 23 | 48 | 97 |
| 12 | 20 | 5 | 20 | Glyme | 4 | 0 | 140 | 20 | 30 | 25 | 43 | 98 |
| 13 | 10 | 5 | 20 | Glyme | 2 | 0 | 140 | 15 | 32 | 19 | 48 | 99 |
| 14 | 30 | 1 | 20 | Glyme | 4 | 0 | 160 | 30 | 29 | 23 | 43 | 95 |
| 15 | 10 | 5 | 20 | THF | 2 | 0 | 140 | 30 | 35 | 9 | 44 | 88 |

### Example 2

13.0 g of HFCS-90 (77.2% DS, 93.7% fructose, 4.1% glucose, 2.2% DP2+), 3.3 mL of 1 M aq. HCl, 12.6 mL of water, and 80.8 mL of dimethoxyethane (DME) were combined in a sealed container and heated with stirring at 120° C. for 60 minutes. On cooling, a sample was taken and analyzed by HPLC for fructose+glucose, reaction intermediates, and HMF. HMF yield (based on total sugars): 48%; sum of unconverted fructose+mol % yield of intermediates+mol % yield of HMF: 99%.

### Example 3

10 g of fructose (56 mmol fructose), 3.3 mL of 1 M aq. HCl (3.3 mmol HCl), 18 mL of water, and 80 mL of dimethoxyethane (DME) were combined in a sealed container and heated with stirring at 150° C. for 65 minutes. The solution was cooled and the DME was removed by vacuum rotary evaporation. To the resulting aq. solution was added 60 mL of methyl isobutyl ketone (MIBK) and the mixture was stirred vigorously and allowed to phase separate. Samples from each layer were taken and analyzed by HPLC for fructose, reaction intermediates, and HMF. HMF yield (based on fructose): 36%; sum of unconverted fructose+mol % yield of intermediates+mol % yield of HMF: 98%. Table 2 reports the distribution of the reaction constituents (fructose, reaction intermediates and HMF) in the different layers (phases).

TABLE 2

| | Volume | Fructose | Intermediates | HMF |
|---|---|---|---|---|
| Layer | (mL) | mol % | mol % | mol % |
| Top | 59 | 0% | 0% | 90% |
| Bottom | 9 | 100% | 100% | 10% |

### Example 4

120 g of fructose (666 mmol fructose), 33 mL of 1 M aq. HCl (33 mmol HCl), 67 mL of 5 M aq. NaCl (333 mmol NaCl), and 400 mL of 2-BuOH (1^{st} solvent) were combined in a sealed container and heated with stirring at 120° C. for 45 minutes. On cooling to room temperature, 50 mL of hexane (2^{nd} solvent) was added, the mixture was stirred vigorously, and allowed to separate. Samples from each layer were taken and analyzed by HPLC for fructose, reaction intermediates, and HMF. HMF yield (based on fructose): 30%; sum of unconverted fructose+mol % yield of intermediates+mol % yield of HMF: 93%. Table 3 reports the mole fractions of reaction constituent (fructose), intermediates and product in the different layers (phases).

TABLE 3

| | Volume | Moles | Moles reaction | Moles |
|---|---|---|---|---|
| Layer | (mL) | fructose | intermediates | HMF |
| Top | 544 | 0.021 | 0.00 | 0.181 |
| Bottom | 170 | 0.259 | 0.141 | 0.022 |

### Example 5

To the bottom layer of Example 4 was added 45 g fructose (242 mmol fructose), 29 mL of 1 M aq. HCl (29 mmol HCl), and 400 mL of 2-BuOH. The mixture was heated with stirring in a sealed container at 120° C. for 45 minutes. On cooling to room temperature, 50 mL of hexane was added, the mixture was stirred vigorously, and allowed to separate. Samples from each layer were taken and analyzed by HPLC for fructose, reaction intermediates, and HMF. HMF yield (based on fructose+reaction intermediates): 32%; sum of unconverted fructose+mol % yield of intermediates+mol % yield of HMF: 93%. Table 4 reports the mole fractions of reaction constituent (fructose), intermediates and product in the different layers (phases).

TABLE 4

| | Volume | Moles | Moles reaction | Moles |
|---|---|---|---|---|
| Layer | (mL) | fructose | intermediates | HMF |
| Top | 585 | 0.027 | 0.00 | 0.210 |
| Bottom | 159 | 0.230 | 0.139 | 0.021 |

### Example 6

In this Example, commercially available acid-functionalized polymeric ion exchange resins were tested for fructose dehydration to HMF using the following catalyst testing protocol.

Catalyst was weighed into a glass vial insert followed by addition of 300-1000 μl of 5 wt % fructose, fructose+glucose and/or Invertose HFCS-90 solution plus solvent (5:1 organic solvent to water). The glass vial insert was loaded into a reactor and the reactor was closed. The atmosphere in the reactor was replaced with nitrogen and pressurized to 300 psig at room temperature. Reactor was heated to 120° C. and maintained at 120° C. for 30-120 minutes while vials were shaken. After the specified reaction time, shaking was stopped and the reactor was rapidly cooled to 40° C. Pressure in the reactor was then slowly released. The solutions were diluted with water and analyzed by liquid chromatography with CAD and UV detection and gas chromatography with flame ionization detection. The particulars of a variety of runs using the catalysts are reported in Table 5. For entries 6, 7 and 9, which utilized solutions comprised of fructose with 10-20% glucose by weight, mol % unconverted fructose reported in Table 5 reflects the amount of fructose+glucose within the reaction solution at time of quench.

TABLE 5

| | | | | | | | | | | | Sum of |
|---|---|---|---|---|---|---|---|---|---|---|---|
| | | | | | | | | | | | unconverted |
| | | | | | | | Run | Unconverted | | | Fructose + |
| | | | H+ | Catalyst | Reaction | | Time | Fructose | Intermediates | HMF | Intermediates + |
| Entry | Substrate | Resin | (meq/g) | (mg) | Volume (ul) | Solvent | (min) | mol % | mol % | mol % | HMF |
| 1 | Fructose | Amberlyst 15 | 4.85 | 10 | 400 | Glyme | 30 | 34 | 10 | 49 | 94 |
| 2 | Fructose | Amberlyst 15 | 4.85 | 9 | 500 | Glyme | 30 | 40 | 12 | 42 | 95 |
| 3 | Fructose | Amberlyst 15 | 4.85 | 9 | 750 | Glyme | 30 | 49 | 12 | 31 | 92 |
| 4 | Fructose | Purolite 275 DR | 4.26 | 10 | 500 | Glyme | 30 | 36 | 11 | 45 | 92 |
| 5 | Fructose | Purolite 275 DR | 4.26 | 4 | 1000 | Glyme | 120 | 50 | 0 | 45 | 95 |
| 6 | Invertose | Purolite 275 DR | 4.26 | 7 | 400 | Glyme | 30 | 44 | 11 | 42 | 97 |
| | HFCS-90 | | | | | | | | | | |
| 7 | Fructose + | Purolite 275 DR | 4.26 | 7 | 400 | Glyme | 30 | 45 | 8 | 39 | 92 |
| | Glucose (4:1) | | | | | | | | | | |
| 8 | Fructose | Purolite 275 DR | 4.26 | 9 | 750 | Glyme | 30 | 48 | 12 | 34 | 94 |
| 9 | Fructose + | Purolite 275 DR | 4.26 | 6 | 600 | Glyme | 30 | 54 | 12 | 30 | 96 |
| | Glucose (9:1) | | | | | | | | | | |
| 10 | Fructose | Purolite 275 DR | 4.26 | 7 | 600 | Glyme | 30 | 49 | 13 | 36 | 97 |
| 11 | Fructose | Purolite 275 DR | 4.26 | 4 | 600 | IPA | 120 | 40 | 2 | 49 | 90 |
| 12 | Fructose | Purolite 275 DR | 4.26 | 11 | 400 | IPA | 30 | 41 | 8 | 42 | 91 |
| 13 | Fructose | Purolite 275 DR | 4.26 | 5 | 1000 | IPA | 120 | 52 | 0 | 37 | 90 |

### Example 7

In this example, high fructose corn syrup was converted to HMF in a continuous flow reactor.

The flow reactor consisted of a 0.25″×73″ zirconium tube having an approximate volume of 30.0 mL. The reactor tube was vertically mounted in an aluminum block heater equipped with PID controller. Feed solutions were delivered in upflow mode using two HPLC pumps and the reactor pressure was controlled at 300 psi by means of a back pressure regulator.

Two feed solutions were prepared, Feed 1: 10 wt % HFCS-90, dissolved in Dioxane/H₂O (4/1 by volume); and Feed 2: 10 wt % HFCS-90, 0.12 wt % HCl dissolved in Dioxane/H₂O (4/1 by volume).

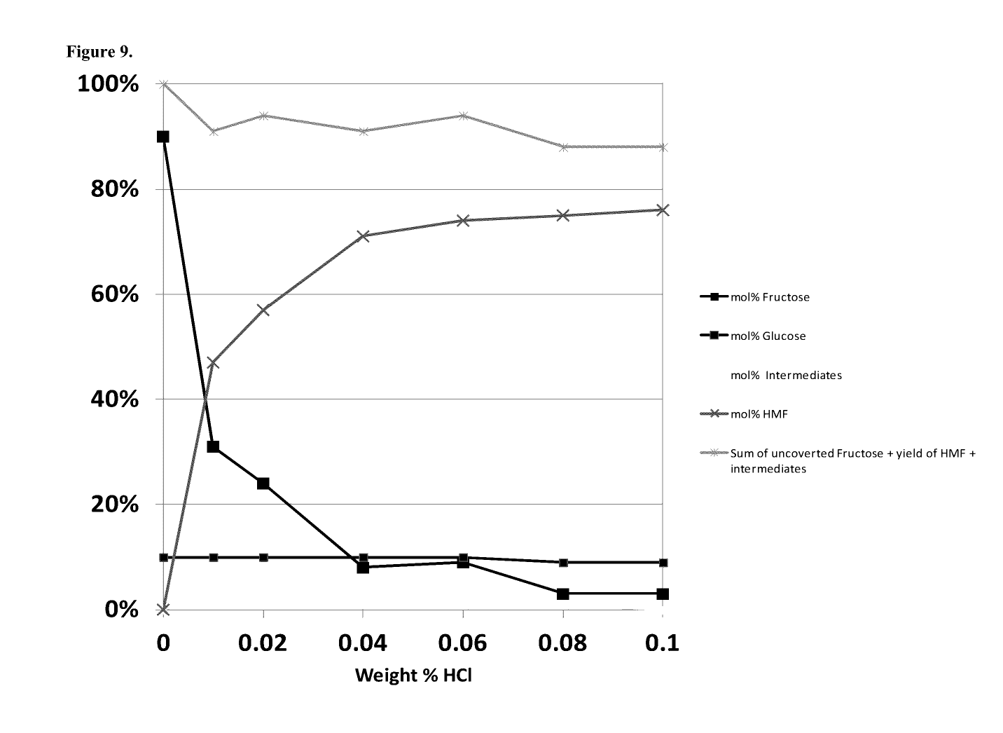

The reaction was performed at 120° C. with a fixed residence time of 5 minutes and a total feed flow rate of 6 mL/min. Reaction conversion was controlled by varying the amount of HCl through changes in the flow ratio of Feed 1 and Feed 2. Reaction progress was monitored and product composition was determined by HPLC analysis on a Thermo Ultimate 3000 analytical chromatography system using a porous graphitic stationary phase (Hypercarb, 3.0×100 mm, 5 um) at 30° C. Fructose and glucose were eluted under isocratic conditions of 0.005% v/v NH₄OH in H₂O at a flow rate of 0.6 mL/min. Intermediates and 5-(hydroxymethyl)furfural (HMF) were eluted by employing a gradient of up to 60% MeOH at a flow rate of 1.0 mL/min. Fructose, glucose and intermediates were detected using a universal charged aerosol detector (CAD) and HMF was detected by UV at 254 nm. Fructose, glucose, and HMF were quantified by fitting to calibration curves generated from pure standards. Intermediates were quantified using a calibration curve generated from a structurally related reference compound. The results are summarized in the Table 6 below and the data from this example is depicted graphically in FIG. 9.

TABLE 6

| | | | | | Sum of mol |
|---|---|---|---|---|---|
| | | | | | fraction % of |
| | | | | | unconverted |
| | | | | | Fructose + |
| | Unconverted | | | | mol % |
| wt % | Fructose | Glucose | Intermediates | HMF | Intermediates + |
| HCl | mol % | mol % | mol % | mol % | mol % HMF |
| 0.00 | 90% | 10% | 0% | 0% | 100% |
| 0.01 | 31% | 10% | 4% | 47% | 91% |
| 0.02 | 24% | 10% | 3% | 57% | 94% |
| 0.04 | 8% | 10% | 2% | 71% | 91% |
| 0.06 | 9% | 10% | 1% | 74% | 94% |
| 0.08 | 3% | 9% | 1% | 75% | 88% |
| 0.10 | 3% | 9% | 0% | 76% | 88% |

### Example 8

In this example, ultra-filtration and nano-filtration membranes were used to remove humins from the aqueous product effluent resulting from conversion of fructose to HMF.

Product effluent for testing of ultra- and nano-filtration was produced under conditions analogous to those described in Example 7, but using 1,2-dimethoxyethane (DME) as the solvent (4/1 DME/water by volume). This partial conversion continuous flow process gave an organic solvent/water product mixture consisting of 24 mol % fructose, 8 mol % glucose, 9 mol % intermediates, 56 mol % HMF and 3 mol % unidentified oligomeric or polymeric materials referred to as humins.

The HCl in the collected product effluent was neutralized with 1 eq of NaOH prior to removal of DME by rotary evaporation. The remaining crude aqueous product mixture was diluted 3.8 times by volume with deionized water and subjected to ultra-filtration and nano-filtration treatment for removal of humins.

In one test, cross-flow ultra-filtration was performed by circulating 2 L of the opaque dark brown aqueous product mixture through a 2.7 m² spiral wound GE UF membrane having a molecular weight cut-off (MWCO) of 1000 available from GE Water & Process Technologies, Inc. After 4.25 minutes, the collected permeate was analyzed by HPLC. Fructose, glucose, HMF, and intermediates all passed through the membrane while a majority of the colored bodies (humins) did not and remained in the retentate. The collected permeate was a clear orange solution.

In another test, cross-flow nano-filtration was performed by circulating 1 L of the opaque dark brown aqueous product mixture through a 2.7 m² spiral wound Dairy NF membrane having a MWCO of 150 available from GE Water & Process Technologies, Inc. After 3.8 minutes, the collected permeate was analyzed by HPLC. The permeate consisted of HMF substantially free of fructose, glucose, intermediates, and colored bodies (humins). The collected permeate was a clear pale yellow solution.

In another test, cross-flow filtration was performed by circulating 1 L of the opaque dark brown aqueous product mixture through a 2.6 m² spiral wound H series membrane having a MWCO of 150-300 available from GE Water & Process Technologies, Inc. After 20.0 minutes, the collected permeate was analyzed by HPLC. The permeate consisted primarily of HMF with a very small amount of fructose and no detectable quantity of glucose or intermediates. The colored bodies (humins) were substantially removed. The collected permeate was a clear pale yellow solution.

### Example 9

In this example, aluminum oxide is used as an adsorbant to remove humins, 2-hydroxyacetylfuran (HAF), and unreacted glucose from an organic solvent/water product effluent resulting from conversion of fructose to HMF. HMF and the by-product furfural are not adsorbed on alumina and can be recovered from the purified solution. The adsorbed by-products can be desorbed from the solid aluminum oxide using aqueous NaOH solution. This allows a facile method for regenerating the adsorbant for use in additional product purification reactions.

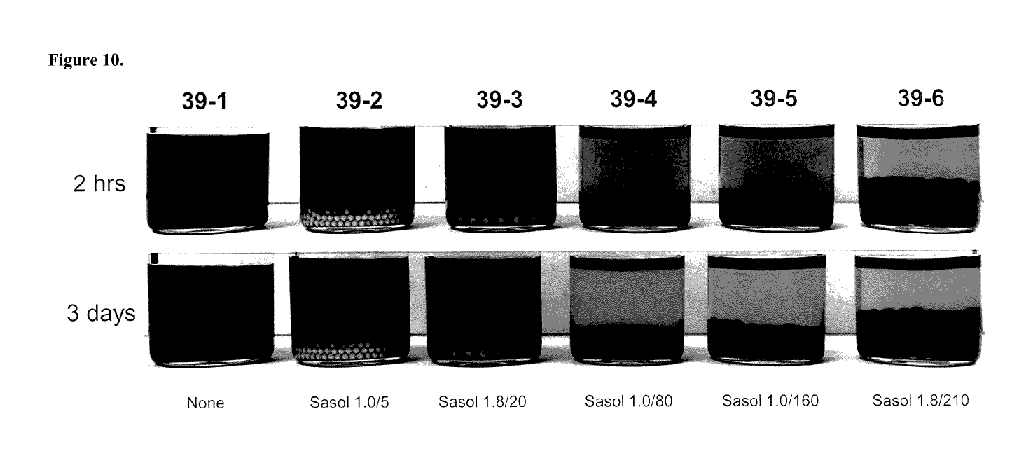

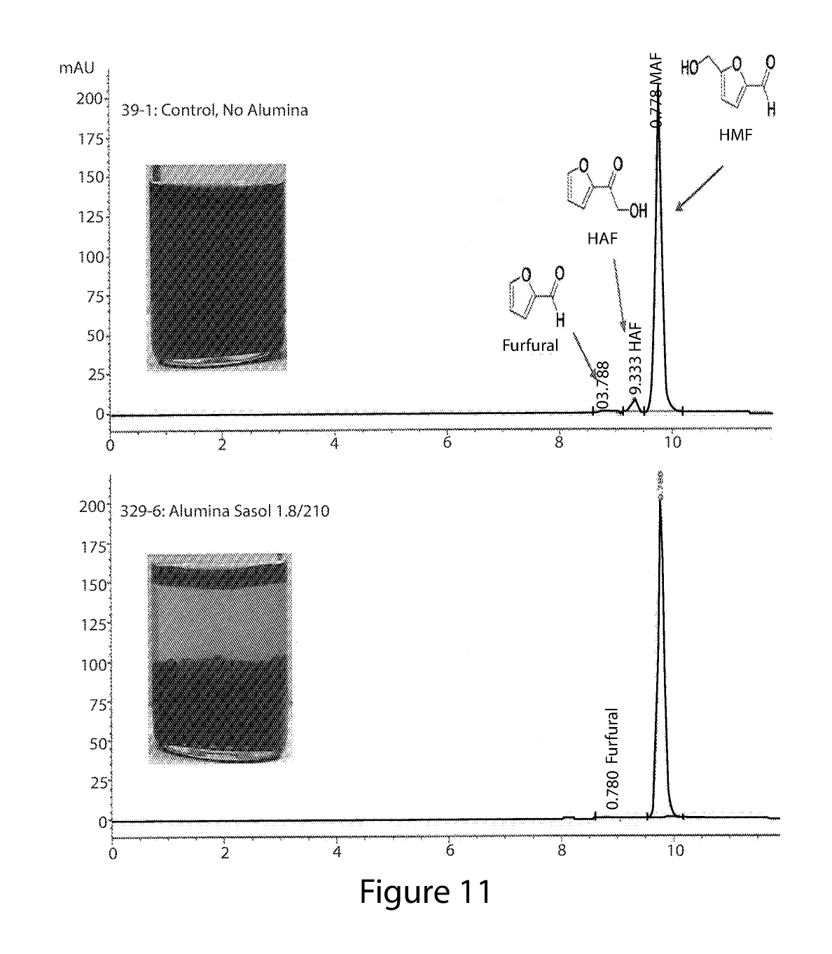

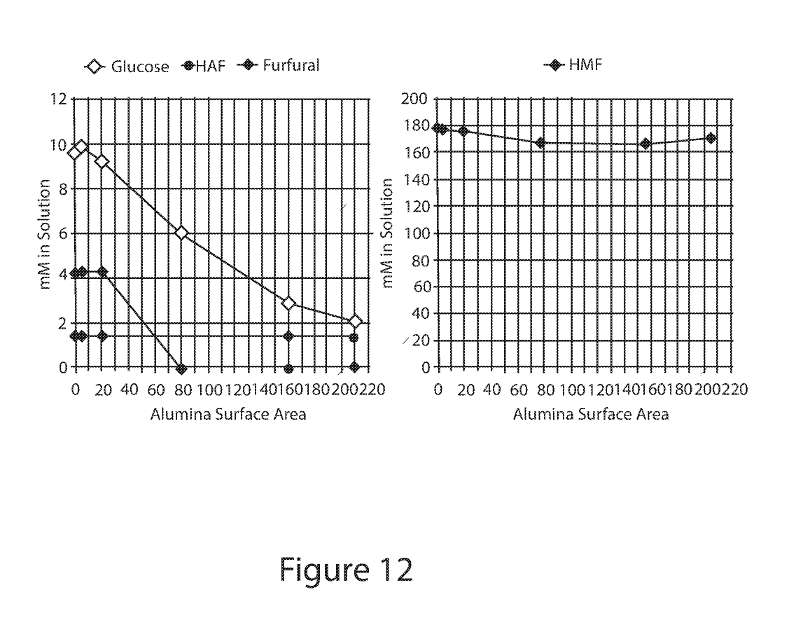

A permeate solution was prepared from a product effluent comprising 5 HFCS (5 wt %), HCl (0.06 wt %) and dioxane/water. 8 mL vials were charged with ˜1.97 g of the permeate solution. Alumina spheres (0.5 g) were added to each solution. A controlled solution was prepared in which no alumina was added. Five different alumina spheres were tested, as shown in Table 7. As illustrated in FIGS. 10-12, the efficacy of solid aluminum oxide as a selective adsorbant is correlated with the surface area and/or crystal phase of the aluminum oxide.

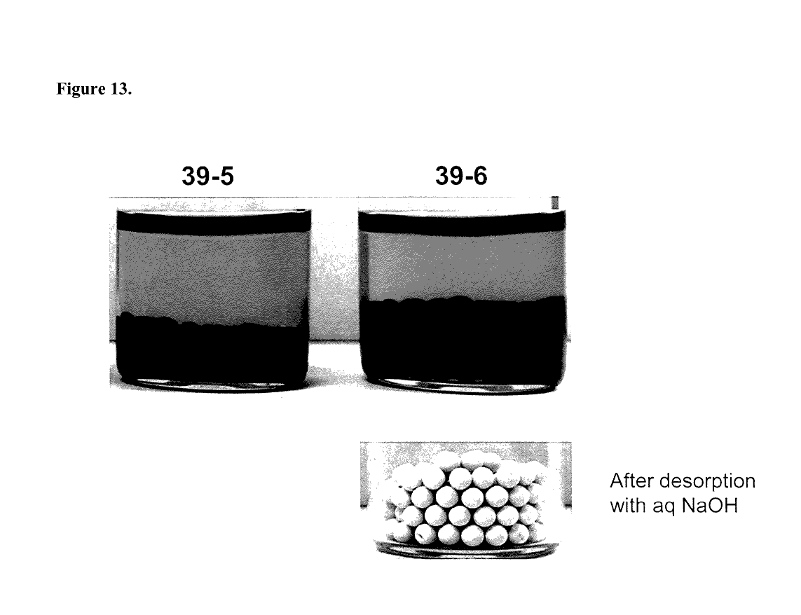

As illustrated in FIG. 13, humins are not significantly desorbed from alumina spheres using dioxane/water, acetone or methanol solutions. When alumina spheres are treated with 0.1N NaOH (aq.), humins are immediately desorbed. Higher surface area alumina spheres (SA≧80) absorb humins (color) without adsorbing HMF or furfural. The HAF by-product is completely adsorbed and removed from solution. These aluminas show high selectivity towards adsorbing humins and HAF over furfural and HMF. Remaining sugars were also scavenged by alumina.

TABLE 7

| | | Sasol | Sasol | Sasol | Sasol | Sasol |
|---|---|---|---|---|---|---|
| Alumina | None | 1.0/5 | 1.8/20 | 1.0/80 | 1.0/160 | 1.8/210 |
| Sphere | NA | 1 | 1.8 | 1 | 1 | 1.8 |
| Diameter | | | | | | |
| (mm) | | | | | | |
| Surface | NA | 5 | 20 | 80 | 160 | 210 |
| Area | | | | | | |
| (cm²/g) | | | | | | |

* Feed Loading = 1.97 g, Alumina Loading = 0.5 g

### Example 10

In this example, aluminum oxide (Sasol 0.5/200) is used as an adsorbant to remove humins, 2-hydroxyacetylfuran (HAF), and unreacted glucose from a reaction solution effluent resulting from conversion of fructose to HMF. 0.28 g Sasol 0.5/200 was added to a 2.6 g reaction solution containing fructose (5.7 wt %; 0.293 M), HCl (0.1 wt %), water (18.5 wt %), and dimethoxyethane (75.7 wt %) at pH 1.81. It was observed that the pH of the solution became more acidic (pH lowered) over time after removal of alumina from the solution. The results from this experiment are shown in Table 8 below.

TABLE 8

| % Conversion | 35 | 67 | 82 | 87 |
|---|---|---|---|---|
| pH | 6.81 | 6.34 | 6.08 | 5.95 |

% Remaining in Solution After Alumina Treatment

| | | | | |
|---|---|---|---|---|
| Fructose | 77.2 | 78.7 | 72.2 | 78.5 |
| DFA Inter | 81.2 | 79.4 | 75.8 | 73.5 |
| Furfural | 100 | 89.9 | 92.2 | 100 |
| HAF | 59.8 | 85.2 | 82.6 | 79.9 |
| HMF | 96.7 | 100 | 100.5 | 100.5 |

### Example 11

In this example, activated carbon (Norit GAC 1240+) is used as an adsorbant to remove humins, 2-hydroxyacetylfuran (HAF), and unreacted glucose from a reaction solution effluent resulting from conversion of fructose to HMF. 0.28 g Norit GAC 1240+ was added to a reaction solution containing fructose (5 wt %), HCl (0.1 wt %), water (18.5 wt %), and dimethoxyethane (76.4 wt %) at pH 1.65. The results of this experiment are shown in Table 9 below.

TABLE 9

| Sample | 73-1 | 73-2 | 73-3 | 73-4 |
|---|---|---|---|---|
| % Conversion | 38 | 65 | 77 | 85 |
| pH | 1.72 | 1.76 | 1.78 | 1.81 |

% Remaining in Solution After Carbon Treatment

| | | | | |
|---|---|---|---|---|
| Fructose | 108 | 104 | 103 | 105 |
| DFA Inter | 102 | 102 | 98 | 108 |
| Furfural | 93 | 88 | 87 | 90 |
| HAF | 66 | 79 | 81 | 84 |
| HMF | 81 | 90 | 88 | 91 |

Some evaporative loss was observed in sample 73-3. Reaction mixtures were decolorized using 0.28 g of Norit GAC 1240+ for 24 h. Solution pH was measured using a pH probe after 20× dilution of the sample in water.

A comparison of the results observed in Examples 10 and 11 show much better color removal with carbon. However, some adsorption of HMF on carbon was observed. The solutions treated with carbon catalyst remain acidic. No adsorption of HMF was observed in samples treated with alumina catalyst. Alumina was found to adsorb HCl from solution. Solution pH increases upon treatment with alumina but drops to pH 3-4 after removal of alumina from the samples. In particular, the solution can be adjusted to pH 7 with alumina. The drop to pH 3-4, and not pH 1-2, after removal of alumina suggest s HCl has been scavenged from solution. It has been determined that a solution of pure HMF has a pH of 3-4. The solution pH remains low (1-2) and essentially unchanged with addition of carbon. This indicates acid is not adsorbed and removed from solution as is the case with alumina

### Example 12

In this Example, HMF is formed by way of an in situ transformation of transient HMF to BHMF.

A heterogeneous solid acid catalyst Purolite 482 was used (supplied by Puriolite, containing a proton loading of 4.82 meq./g). A heterogeneous reduction catalyst comprising 1 wt. % Pt supported on Fe₂O₃ was prepared as follows: 0.5 g of Fe₂O₃ (supplied by Baker) was impregnated with a 0.25 mL of a solution prepared by combining 0.08 mL of a solution of (NH₃)₄Pt(OH)₂ (63.2 mg/ml Pt) with 0.17 mL of deionized water. The resultant material was dried at 120° C. for 2 hours, calcined at 300° C. for 4 hours and then reduced under a flow of forming gas at 350° C. for a further 3 hours.

200 μL of a 5/1 diglyme/water (volumetric ratio) mixture containing 10 wt. % fructose was dispensed into 2×1 ml glass vials along with 20 mg of a solid acid catalyst. Into one of the glass vials was added 20 mg of the heterogeneous reduction catalyst. The second glass vial was left without a reduction catalyst as a control. The glass vials were loaded into reactor pressure vessel and sealed. The reactor was pressurized to 300 psi with hydrogen and heated to a temperature of 120° C. with orbital shaking for 3 hours. After 3 hours, the shaking was stopped and the temperature was brought down to room temperature and the pressure was slowly released. The glass vial was removed and a reaction aliquot was sampled for analysis by both GC and HPLC. The reaction product from the control vial was a dark brown solution indicative of the presence of humins. The principal products detected by GC and HPLC from the control vial were HMF and levulinic acid. The reaction product from the vial containing the reduction catalyst was a light orange color indicative of lower levels of humins. The principal reaction products detected by GC and HPLC were BHMF and HMF, demonstrating that BHMF can be prepared by in situ reduction of HMF prepared from the catalytic dehydration of fructose.

### Example 13

This example describes a dehydration reaction, converting fructose to HMF using tetraethylammonium bromide. An aqueous stock solution was prepared by combining Fructose (1.2 g, 6.66 mmol), tetraethylammonium bromide (0.6 g, 2.86 mmol), H₂O (3.8 g) and 1N aqueous HCl (0.669 g). A glass reaction vial was charged with 0.786 g of this aqueous solution followed by addition of 1,4-dioxane (2.23 g). The resulting homogeneous monophasic reaction solution was sealed and heated at 120° C. for 1 h with mechanical stirring. After cooling to room temperature, an aliquot was removed and diluted with H₂O for HPLC analysis. Quantification of fructose and HMF was made using external calibration standards. The molar yield of HMF was 82.5% at 98% Fructose conversion.

### Example 14

This example describes a dehydration reaction, converting fructose to HMF without using tetraethylammonium bromide (comparison example). An aqueous stock solution was prepared by combining Fructose (1.2 g, 6.66 mmol), H₂O (3.8 g) and 1N aqueous HCl (0.669 g). A glass reaction vial was charged with 0.711 g of this aqueous solution followed by addition of 1,4-dioxane (2.33 g). The resulting homogeneous monophasic reaction solution was sealed and heated at 120° C. for 1 h with magnetic stirring. After cooling to room temperature, an aliquot was removed and diluted with H₂O for HPLC analysis. Quantification of fructose and HMF was made using external calibration standards. The molar yield of HMF was 77.8% at 98% Fructose conversion.

### Example 15

This example describes an experiment in which an ultrafiltration ceramic membrane is used to remove humins from a reaction solution containing HMF, fructose, glucose, and humins, in a solvent combination containing water and a water miscible organic solvent using H₂SO₄ as a catalyst. For this experiment, a ceramic PFM ultrafiltration membrane module with a 800 Dalton molecular weight cut off (MWCO) supplied by Cerahelix was used (see www.cerahelix.com; website Apr. 9, 2016). The membrane was housed in a 1 meter multi-channel filter tube developed by Cerahelix with a usable surface area of 0.18 m² and was deployed with a cross-flow velocity of 10 cms⁻¹ and an applied pressure of 160-200 psi. The separation started with 2.575 L of a reaction product generated from a 10 wt. % solution (in 4/1 v/v Dioxane/H₂O) of high fructose corn syrup 90 (HFCS-90) in which 90% of the fructose was converted using H₂SO₄ as a dehydration catalyst (0.4 wt %) to a darkly colored reaction mixture comprising HMF, humins, unconverted fructose and unconverted glucose. 2.266 L of the reaction solution (without neutralizing the H₂SO₄) was filtered through the ultrafiltration membrane (equal to 88% single pass recovery). UV-Visible spectroscopic analysis of the permeate material revealed that humins were retained by the membrane, while HMF fructose, glucose permeated through the membrane. The purification could be confirmed visually (concentrated humins forms a dark colored solution). This experiment demonstrated the viable use of a ceramic membrane for the separation of HMF from humins in the reaction product at low pH using a solvent composition containing water and a water miscible organic solvent without the need to first remove the organic solvent.

### Example 16

This example describes an experiment in which membrane separations using polymeric membranes were conducted using a cross-flow filtration through a flat sheet membrane (surface are 42 cm²) deployed in a cross-flow membrane testing cell supplied by Sterlitech (see www.sterlitech.com website Apr. 9, 2016). Unless otherwise stated, a cross-flow velocity of 12 cms⁻¹ was used with an applied pressure of 400 psi across the membrane.

The testing cell was used to test 17 polymer membranes in the following manner: A darkly colored reaction product was generated from a 10 wt. % solution (in 4/1 v/v Dioxane/H₂O) of HFCS-90 in which all of the fructose was converted using a very low concentration of HCl as a dehydration catalyst (0.01 wt. %) to a reaction mixture comprising HMF, humins, and glucose. The reaction product was first neutralized by the addition of NaOH (1 stoichimetric equivalent with respect to the HCl present in the starting solution). A cross-flow velocity of 12 cms⁻¹ was used with an applied pressure of 100-400 psi across the membrane within the membrane testing cell. Membranes were tested for compatibility with the reaction solvent by measuring the flux of the permeation. Membranes that showed very low or no measurable flux were considered incompatible. Membranes that showed very high fluxes with no separation were also considered incompatible. The ability of the membrane to reject humins which was assessed by the color of the permeate solution and/or UV/Vis spectroscopy. Membranes capable of humins removal produced a permeate solution that was light yellow or light orange in color, and a retentate solution that was darker in color than in the reaction product. A list of the membranes tested and the results are shown in Table 10 below.

TABLE 10

| Membrane | Membrane | Solvent | Humins |
|---|---|---|---|
| Supplier | Grade | Compatible | Rejection |
| Borsig | GMT_oNF_1 | Yes | Yes |
| Borsig | GMT_oNF_2 | Yes | Yes |
| Borsig | GMT_NC_1 | Yes | Yes |
| AMS Technologies | U301 | Yes | Yes |
| AMS Technologies | 3014 | Yes | Yes |
| PolyAn | Pol_oNF_M1_1 | Yes | Yes |
| PolyAn | Pol_oNF_M1_2 | Yes | Yes |
| SolSep | NF010206S | Yes | No |
| SolSep | NF010306S | No | — |
| SolSep | NF080105 | Yes | Yes |
| Evonik | DuraMem 150 | No | — |
| Evonik | DuraMem 200 | No | — |
| Evonik | DuraMem 300 | Yes | Yes |
| Evonik | DuraMem 500 | Yes | Yes |
| Evonik | DuraMem 900 | Yes | No |
| Evonik | PuraMem 280 | Yes | Yes |
| Evonik | PuraMem S600 | No | — |

The results of this experiment demonstrate that a variety of commercially available membrane grades are effective for the removal of humins from the reaction product comprising HMF and a solvent composition containing water and a water miscible organic solvent without the need to first remove the organic solvent. HPLC was used to determine that membrane grades that were deemed solvent compatible and effective for humins removal also allowed HMF to permeate through the membrane.

### Example 17

In this experiment the PolyAn supplied membrane: PolAn_Pol_oNF_M1_2 (see www.poly-an.de website Apr. 8, 2016) was secured in place in the testing cell. The separation started with 3.842 L of a darkly colored reaction product generated from a 10 wt. % solution (in 4/1 v/v Dioxane/H₂O) of HFCS-90 in which almost all of the fructose was converted using a very low concentration of HCl (0.01 wt. %) as a dehydration catalyst. The reaction product was first neutralized by the addition of NaOH (1 stoichimetric equivalent with respect to the HCl present in the starting solution). 3.428 L of the neutralized reaction solution was filtered through the ultrafiltration membrane (90% single pass recovery). HPLC and UV-Visible Spectroscopic analysis of the retentate and permeate materials revealed that humins, and glucose were retained by the membrane, while HMF permeated through the membrane. This experiment demonstrated the viable use of a polymer membrane for the separation of HMF from the reaction product using a solvent composition containing water and a water miscible organic solvent without the need to first remove the organic solvent.

### Example 18

In this experiment, the testing cell described in Example 16 was used to test 7 polymer membranes in the following manner: A darkly colored reaction product was generated from a 10 wt. % solution (in 4/1 v/v Dioxane/H₂O) of HFCS-90 in which all of the fructose was converted using a very low concentration of HCl as a dehydration catalyst (0.01 wt. %). The reaction product was first neutralized by the addition of Ca(OH)₂ (1 stoichimetric equivalent with respect to the HCl present in the starting solution). A cross-flow velocity of 12 cms⁻¹ was used with an applied pressure of 400 psi across the membrane within the membrane testing cell. The ability of the membrane to reject humins was assessed by the color of the permeate solution and/or UV-Visible spectroscopy. Membranes capable of humins removal produced a permeate solution that was light yellow or light orange in color, and a retentate solution that was darker in color than in the reaction product. In all cases, humins were rejected by the membranes while HMF permeated through the membrane. The ability of the membrane to reject the calcium salt was determined by ICP by measuring the calcium concentration of the permeate solution relative to the neutralized reaction product. The calcium content of the retentate was consistent with the expected value from the neutralization. In all cases the calcium content of the permeate was consistent with background calcium concentrations. The results are shown in Table 11.

TABLE 11

| Membrane | Membrane | Humins | Calcium |
|---|---|---|---|
| Supplier | Grade | Rejection | Rejection |
| Borsig | GMT_oNF_1 | Yes | Yes |
| PolyAn | Pol_oNF_M1_1 | Yes | Yes |
| PolyAn | Pol_oNF_M1_2 | Yes | Yes |
| AMS Technologies | U301 | Yes | Yes |
| SolSep | NF080105 | Yes | Yes |
| Evonik | DuraMem 300 | Yes | Yes |
| Evonik | DuraMem 500 | Yes | Yes |

This experiment demonstrated the viable use of a polymer membrane for the separation of HMF from the neutralized reaction product formed by the dehydration of fructose using a solvent composition containing water and a water miscible organic solvent without the need to first remove the organic solvent. After neutralization, rejection of both the unwanted humins by product and the calcium salt by the membrane enables a simple and effective process for the separation of HMF from the catalyst and unwanted humins byproduct thereby producing a permeate stream containing HMF.

When introducing elements of the present invention or the preferred embodiments(s) thereof, the articles “a”, “an”, “the” and “said” are intended to mean that there are one or more of the elements. The terms “comprising”, “including” and “having” are intended to mean that there may be additional elements other than the listed elements.

In view of the above, it will be seen that the several objects of the invention are achieved and other advantageous results attained.

As various changes could be made in the above processes and products without departing from the scope of the invention, it is intended that all matter contained in the above description and shown in the accompanying drawings shall be interpreted as illustrative and not in a limiting sense.

1-74. (canceled)

75. A process for the production of 2,5-bis-hydroxymethylfuran (BHMF) and/or 2,5-bis-hydroxymethyltetrahydrofuran (BHMTHF), the process comprising:
dehydrating fructose to afford 5-(hydroxymethyl)furfural (HMF) in the presence of a dehydration catalyst;
reducing HMF to form BHMF in the presence of a hydrogenation catalyst; and
optionally converting BHMF to form BHMTHF.

76. The process of claim 75, wherein said process is performed in the presence of a solvent.

77. The process of claim 76, wherein said solvent comprises water.

78. The process of claim 76, wherein said solvent comprises water and an immiscible organic solvent.

79. The process of claim 76, wherein said solvent comprises water and a water-miscible organic solvent.

80. The process of claim 75, wherein the rate of the dehydration step is slower than the rate of the reducing step.

81. The process of claim 75, wherein the hydrogenation catalyst is a homogenous catalyst.

82. The process of claim 75, wherein the hydrogenation catalyst is a heterogeneous catalyst.

83. The process of claim 75, wherein the dehydration catalyst is a mineral acid in a single solvent or a single-phase solvent mixture.

84. The process of claim 75, wherein the reaction temperature for the reduction step is the same as the reaction temperature used for dehydration step.

85. A process for the production of HMF comprising the steps of:
converting fructose to HMF;
simultaneously protecting the aldehyde group on HMF with an aldehyde protecting group;
optionally deprotecting the aldehyde-protected HMF to afford HMF.

86. The process of claim 85, wherein said aldehdye protecting group is selected from the group consisting of: cyclic acetals; acyclic acetals, cyanohydrins, hydrazones, oximes, 1,2-adducts with cyclic and acyclic amine adducts, enolate anions, enol ethers, enamines and imines.

87. A process for the production of HMF comprising the steps of:
providing fructose in its furanose form;
converting the furanose form of fructose to form HMF.

88. The process of claim 87, wherein said furanose form of fructose is the β-furanose form.

89. The process of claim 87, wherein said furanose form is provided at room temperature.

90. The process of claim 87, wherein said furanose form of fructose is converted to HMF by way of a dehydration reaction.

91. The process of claim 87, wherein said process is carried out at 120° C.

92. A process for the production of HMF comprising the steps of:
converting fructose to form HMF in a solvent, wherein said fructose is provided in particulate form.

93. The process of claim 92, wherein the solubility of the fructose particles in the solvent is in the less than about 5 wt % to about 10 wt %.

94. The process of claim 92, wherein said solvent comprises a Lewis acid or Bronsted acid catalyst.

95. A process for purifying HMF comprising filtering a reaction mixture comprising HMF over an ultrafiltration ceramic or polymeric membrane, wherein said reaction mixture comprises an organic solvent.

96. A process for removing contaminants from a reaction product comprising HMF the process comprising:
contacting a complex mixture comprising HMF, an undesired sugar dehydration by-product, and a solvent with an adsorbant, said adsorbant comprising a material selected from the group consisting of alumina and a carbon-based material,
said contacting performed for a time period sufficient to produce a product mixture having a concentration of the undesired sugar dehydration by-product that is lower than the concentration of the sugar dehydration by-product in the complex mixture.
